(19) United States
(12) Patent Application Publication
(10) Pub. No.: US 2013/0045542 A1
(43) Pub. Date: Feb. 21, 2013

(54) NEW FORMULATIONS FOR DIAGNOSIS OF ALZHEIMER'S DISEASE

(71) Applicants: Hermann Esselmann, Goettingen (DE); Teja W. Groemer, Ochsenfurt (DE); Johannes Kornhuber, Nuernberg (DE); Piotr Lewczuk, Roettenbach (DE); Juan Manuel Maler, Erlangen (DE); Jens Wiltfang, Essen (DE)

(72) Inventors: Hermann Esselmann, Goettingen (DE); Teja W. Groemer, Ochsenfurt (DE); Johannes Kornhuber, Nuernberg (DE); Piotr Lewczuk, Roettenbach (DE); Juan Manuel Maler, Erlangen (DE); Jens Wiltfang, Essen (DE)

(73) Assignees: UNIVERSITAET DUISBURG-ESSEN, ESSEN (DE); UNIVERSITAETSKLINIKUM ERLANGEN, ERLANGEN (DE); ZENTRALINSTITUT FUER SEELISCHE GESUNDHEIT, MANNHEIM (DE); FRIEDRICH-ALEXANDER-UNVERSITAET ERLANGEN- NUERNBERG, ERLANGEN (DE)

(21) Appl. No.: 13/639,866

(22) PCT Filed: Apr. 7, 2011
(86) PCT No.: PCT/EP2011/001724

§ 371 Date: Oct. 8, 2012

(30) Foreign Application Priority Data
Apr. 9, 2010 (DE) 102010014577.7
Apr. 12, 2010 (DE) 102010014684.6

(51) Int. Cl. G01N 33/566

(52) U.S. Cl. 436/501

(57) ABSTRACT

A method of using at least one quantitative ratio of two different amyloid beta-peptides in a sample of a body fluid from a patient for determining the patient's probability of contracting Alzheimer's disease (AD) or for determining the patient's suffering from a precursor of Alzheimer's disease includes obtaining the sample of the body fluid from the patient. The at least one quantitative ratio is calculated from the two different amyloid beta-peptides from the sample. The patient's probability of contracting Alzheimer's disease (AD) or the patient's suffering from a precursor of Alzheimer's disease is calculated using the at least one quantitative ratio. The two different amyloid beta-peptides are selected from (a) Aβ(1-42), (b) Aβ(2-40) and (c) Aβ(2-42). The at least one quantitative ratio is selected from (a) Aβ(1-42)/(b) Aβ(2-40), (a) Aβ(1-42)/(c) Aβ(2-42), (b) Aβ(2-40)/(a) Aβ(1-42) and (c) Aβ(2-42)/(a) Aβ(1-42).

### CROSS REFERENCE TO PRIOR APPLICATIONS

This application is a U.S. National Phase application under 35 U.S.C. §371 of International Application No. PCT/EP2011/001724, filed on Apr. 7, 2011 and which claims benefit to German Patent Application No. 10 2010 014 577.7, filed on Apr. 9, 2010, and to German Patent Application No. 10 2010 014 684.6, filed on Apr. 12, 2010. The International Application was published in German on Oct. 13, 2011 as WO 2011/124376 A1 under PCT Article 21(2).

### FIELD

The present invention relates to the field of dementia and in particular to the field of neurodegenerative disorders, the principal focus of the present invention being on Alzheimer's disease.

More particularly, the present invention relates to the field of the preclinical and clinical precursors of Alzheimer's disease.

The present invention relates more particularly to a novel neurochemical approach or novel neurochemical analysis of particular parameters in a sample from the subject or patient to be examined, wherein a significantly improved prognosis with regard to the occurrence or development of Alzheimer's disease is enabled or the presence of Alzheimer's disease or the preclinical and clinical precursors thereof can be concluded.

More particularly, the present invention relates to the use of a quantitative ratio of two different amyloid beta-peptides in a sample of a body fluid from a subject or a patient for determining his or her probability of contracting Alzheimer's disease, or for determining his or her suffering from a precursor of Alzheimer's disease.

In addition, the present invention relates to corresponding methods with which it is possible to determine the probability of a subject or patient contracting Alzheimer's disease, or with which the presence of such disease can be concluded.

Finally, the present invention relates to a kit which provides means of determining the probability of a patient or subject contracting Alzheimer's disease, or of determining the presence of a preclinical or clinical precursor of Alzheimer's disease in the subject or patient in question.

### BACKGROUND

Dementia is generally understood to mean a deficit in the subjects or patients in question in relation to cognitive, emotional and social skills, which leads to sometimes severe impairment of the daily life of the individuals affected as the disease advances. A crucial feature in dementia is the loss of skills already acquired, and this can affect particularly language, motor skills and the personality structure. Dementia is generally associated with a distinct deterioration in short-term memory and severe deterioration in intellectual capacity.

A major proportion of dementia is accounted for by what are called neurodegenerative disorders, such as Alzheimer's disease as discussed, frontotemporal dementia and Lewy body dementia.

Neurodegenerative disorders are generally understood to mean a group of usually slow-advancing disorders of the central nervous system which has hereditary or sporadic origins or causes. A characteristic feature of such disorders is the advancing loss of nerve cells, which can lead to various neurological symptoms. Neurodegenerative disorders can in principle occur at all stages of life, although the risk of contracting such a disorder increases significantly with increasing age. Neurodegenerative disorders generally have a diffuse or generalized course and are generally each accompanied by characteristic histological damage patterns.

One of the most common forms of neurodegenerative disorders is that of Alzheimer's disease, which is generally and also hereinafter abbreviated by the acronym “AD”. Alzheimer's disease is likewise a neurodegenerative disorder, which occurs especially in older people. Thus, Alzheimer's disease in its most common form occurs in people over the age of 65, although the corresponding precursor forms of Alzheimer's disease or preclinical and/or clinical precursors of Alzheimer's disease can also occur much earlier.

The prevalence of neurodegenerative disorders and especially of Alzheimer's disease is increasing significantly. Thus, the number of people affected by Alzheimer's disease at the end of 2009 was estimated to be about 35 million people globally, and it is estimated in this regard that the number of people affected by Alzheimer's disease will rise to more than 100 million people by 2050.

The prevalence of neurodegenerative dementia is constantly increasing globally, as a result of which public health systems in the future will be subject to severe socio-economic stresses. In this context, Alzheimer's disease is the most common type of dementia.

With regard to the causes and the distribution of Alzheimer's disease, a certain degree of familial clustering is found, in that about 15% to 20% of the Alzheimer's cases detected being associated with a particular familial cluster. Such cases are especially those in which, as well as the affected person him- or herself, people from the group of brothers and sisters, parents and grandparents etc. are also affected by Alzheimer's disease. Only in the case of a very small number of sufferers is a genetic predisposition also important, this involving particularly mutations in presenilin genes and in the APP gene (amyloid precursor gene). In relation to the total cases of Alzheimer's disease, however, the genetically related proportion is less than 0.5%, and so the conclusion is that the much greater portion of all Alzheimer's cases is of sporadic origin.

Another feature of Alzheimer's disease as such is that it is associated with a deterioration in cognitive performance which increases over a time of suffering and is generally associated with a decrease in daily activity, peculiar behavior and neuropsychological symptoms. In this context, reference is generally also made to Alzheimer's dementia. Alzheimer's dementia is to some degree the consequence or the result of Alzheimer's disease, describing the progression of the disease. Reference is thus made primarily to Alzheimer's dementia when the degree of brain damage is so great that cognitive deficits can no longer be compensated for successfully by neurons which are still intact. In general, the term “Alzheimer's dementia” is covered by the term “Alzheimer's disease”.

Pathognostic features of Alzheimer's disease are the plaques which form in the brain of the sufferer, these crucially featuring incorrectly folded amyloid beta-peptides, and the neurofibrils which accumulate in the neurons. The intracellular neurofibril bundles consist essentially of the tau protein, which then aggregates to form the fibrils in question when excessively phosphorylated.

In the course of the disease, the death of neurons results in manifestation of brain atrophy; moreover, the acetylcholine messenger is no longer produced in sufficient amounts, a particular cause of which is also a reduction in the content of choline acetyltransferase.

The amyloid beta-peptides, which are also referred to synonymously as A beta-proteins, A beta-peptides, A-beta or Aβ peptides, form from the amyloid precursor protein (APP), which is an integral membrane protein. APP is a type I transmembrane protein with an amino-terminal end present on the extracellular side. The carboxyl terminus of the protein is accordingly on the intracellular side. APP is cleaved by particular proteinases, called the secretases, and particularly β-secretase and γ-secretase are of particular importance here. The cleavage of APP results in the release of the A beta-peptides from the precursor protein, with cutting of APP by β-secretase on the extracellular side in a first step in the course of what is called the amyloidogenic pathway. In a subsequent step, a further cut is effected by γ-secretase, which is effected within the transmembrane domain of APP and leads to release of the A beta-peptides.

The A beta-peptides are thus a potentially neurotoxic fragment formed from the amyloid precursor protein as a result of the activity of β- and γ-secretase, this generally having a length of 37 to 42 amino acids. The main type of A beta-peptides formed in this context is Aβ(1-40) having a length of 40 amino acids, while a smaller proportion of Aβ(1-42) correspondingly having 42 amino acids is formed. The longer Aβ(1-42) has a higher tendency to aggregation than the smaller Aβ(1-40). In addition, a number of further A beta-peptides can form, for example, A beta-peptides truncated at the amino-terminal end, such as Aβ(2-40) and Aβ(2-42).

In the prior art, A beta-peptides are connected to the pathological development of Alzheimer's disease.

Against the above background, attempts have been made in the prior art to implement numerous therapeutic approaches, which are intended to enable therapeutic treatment of Alzheimer's disease per se. Thus, one area of therapeutic focus is that of achieving immunization with respect to the above-described A beta-peptides, especially in connection with the inducement of biosynthesis of antibodies which are specific in this regard. A further therapeutic approach consists in the development and use of specific secretase inhibitors, which are especially intended to be specific with regard to the aforementioned β-secretase or γ-secretase. However, the approaches in this regard are still not satisfactory from a therapeutic point of view or are associated with sometimes serious side effects.

A further therapeutic approach is that of the controlled administration of acetylcholine esterase inhibitors, which are supposed to reduce the enzymatic cleavage of acetylcholine, which occurs in excessively small amounts in Alzheimer's sufferers. However, this is not a therapeutic approach which influences the disease per se. This is because the aim of administering acetylcholine esterase inhibitors such as donepezil, rivastigmine and galantamine, is a symptomatic treatment of Alzheimer's disease. On the basis of such an approach, it is possible merely to slightly improve the cognitive performance for a limited time, or to keep it at a stagnant level.

In conclusion, no therapeutic approach has been found in the prior art to date which can be used to heal Alzheimer's disease. Especially also against the background of an effective therapeutic approach for treatment of Alzheimer's disease, which has been lacking to date, very early diagnosis of the disease is of very great importance, and the diagnosis should be made at an early clinical or even preclinical stage of Alzheimer's disease. This is because it is firstly possible on this basis not just to make a prognosis or determine a probability of whether a subject or patient could contract Alzheimer's disease, but it is secondly also possible on this basis to counteract the further progression of the disease at a very early stage. For instance, it is generally acknowledged that, for example, regular movement, targeted memory exercises and targeted optimization of lifestyle can delay or alleviate the course of the disease, and can delay the actual outbreak, i.e., the clinical diagnosis of Alzheimer's disease. Very early diagnosis of Alzheimer's disease, which already sets in at the level of preclinical or early clinical precursors, is also of great significance with regard to new therapeutic approaches for treatment of Alzheimer's disease.

Against this background, there have already been studies in the prior art of numerous approaches which are intended to enable very early diagnosis of Alzheimer's disease. For instance, in this context, generally established clinical methods are of significance, these being based crucially on differential diagnosis, including specially designed cognitive performance tests and also heteroanamnesis, i.e., the consideration of observations from the family members of an affected person. Imaging methods, such as computer tomography or magnetic resonance tomography, are also employed in order to enable diagnosis of Alzheimer's disease. However, the aforementioned approaches are afflicted with the disadvantage that they firstly sometimes involve complex apparatus and generally do not enable diagnosis of Alzheimer's disease until a later or advanced clinical stage.

A further approach to the early diagnosis of Alzheimer's disease involves analysis of liquor from a subject or patient, particularly employing cerebrospinal fluid (CSF) for the analysis. This principally targets the analysis of particular biomarkers, also with reference in this context to specific A beta-peptides which occur in the liquor for the analysis. A disadvantage is that the sample, however, has to be taken via a complex puncture which is not risk-free and is sometimes painful, and which can sometimes also be associated with unwanted infections. Thus, liquor sampling is only of limited suitability for investigation in the event of a first suspicion of possible Alzheimer's disease, as can exist, for example, in patients with slight cognitive impairment, and also only of limited suitability for estimating the risk of impending Alzheimer's dementia.

The prior art has identified specific biomarkers for dementia in the cerebrospinal fluid, as a result of which the diagnostic accuracy of reliable detection of Alzheimer's disease at an early or even preclinical stage is said to be improved to a certain degree. Compared to blood analyses, the neurochemical CSF diagnosis methods for recognition of dementia (CSF-Based Neurochemical Dementia Diagnostics (CSF-NDD)) are highly invasive and additionally more painful, more expensive and more time-consuming. Routine repeat analyses for the purpose of follow-up checks therefore cannot be conducted.

There is consequently a great need for alternative test methods with simplified and less stressful sampling, which should additionally also enable the monitoring of the course of the disease or of therapeutic success.

### SUMMARY

Against the background cited above, an aspect of the present invention is to provide novel and efficient concepts with regard to specific processes and uses, on the basis of which a statement is to be enabled as to the presence of Alzheimer's disease or a prognosis with regard to possible contraction of Alzheimer's disease, at a very early, especially at a preclinical or early clinical stage.

More particularly, in the context of the present invention, a corresponding concept or method is to be provided, which is easy to perform and is associated with a minimum level of side effects. At the same time, in the context of the present invention, it should also be provided that the provision or taking of samples to be analyzed for the purposes of the inventive concept is associated with a minimum level of pain for the subjects and with a minimum level of side effects, if any.

A further aspect of the present invention is to provide a kit which can be used to enable simple and standardized performance of the inventive concept for determination of Alzheimer's disease, especially with regard to the early forms of the disorder discussed.

A further aspect of the present invention is to provide a method and a use and a kit of the aforementioned type, each of which is capable of avoiding or at least reducing the disadvantages of the prior art.

For example, in the context of the present invention, specific biomarkers from a specific sample are to be used in a controlled manner, in order on this basis to enable a very early statement with regard to the presence or any development of Alzheimer's disease.

F, on the basis of the present invention, the intention is also to open up the possibility of, in a simple and safe manner, observing the course of Alzheimer's disease which has already become established or understanding the efficacy of therapeutic approaches.

In an embodiment, the present invention provides a method of using at least one quantitative ratio of two different amyloid beta-peptides in a sample of a body fluid from a patient for determining the patient's probability of contracting Alzheimer's disease (AD) or for determining the patient's suffering from a precursor of Alzheimer's disease which includes obtaining the sample of the body fluid from the patient. The at least one quantitative ratio is calculated from the two different amyloid beta-peptides from the sample. The patient's probability of contracting Alzheimer's disease (AD) or the patient's suffering from a precursor of Alzheimer's disease is calculated using the at least one quantitative ratio. The two different amyloid beta-peptides are selected from (a) Aβ(1-42), (b) Aβ(2-40) and (c) Aβ(2-42). The at least one quantitative ratio is selected from (a) Aβ(1-42)/(b) Aβ(2-40), (a) AB (1-42)/(c) Aβ(2-42), (b) Aβ(2-40)/(a) Aβ(1-42) and (c) Aβ(2-42)/(a) Aβ(1-42).

In an embodiment, the present invention provides a method for determining a probability of a patient contracting Alzheimer's disease (AD) which includes determining at least one quantitative ratio of two different amyloid beta-peptides in a sample of a body fluid from the patient. The two different amyloid beta-peptides are selected from (a) Aβ(1-42), (b) Aβ(2-40) and (c) Aβ(2-42). The at least one quantitative ratio is formed from (a) Aβ(1-42)/(b) Aβ(2-40), (a) Aβ(1-42)/(c) Aβ(2-42), (b) Aβ(2-40)/(a) Aβ(1-42) and (c) Aβ(2-42)/(a) Aβ(1-42).

In an embodiment, the present invention provides a kit for determining a probability of a patient contracting Alzheimer's disease (AD) or for determining the patent's suffering from a precursor of Alzheimer's disease which includes components for determining a quantitative ratio of two different amyloid beta-peptides in a sample of a body fluid from the patient. The components are selected so as to provide a quantitative determination of two different amyloid beta-peptides selected from (a) Aβ(1-42), (b) Aβ(2-40) and (c) Aβ(2-42) so as to determine at least one quantitative ratio selected from (a) Aβ(1-42)/(b) Aβ(2-40), (a) Aβ(1-42)/(c) Aβ(2-42), (b) Aβ(2-40)/(a) Aβ(1-42) and (c) Aβ(2-42)/(a) Aβ(1-42).

### BRIEF DESCRIPTION OF THE DRAWINGS

The present invention is described in greater detail below on the basis of embodiments and of the drawings in which:

FIG. 1: shows an illustrative two-dimensional separation and labeling of A beta-peptides in blood based on a photograph of a 2D-Aβ-WIB;

FIG. 2 shows a freely selected designation or numbering of a total of 18 amyloid beta-peptides separated by means of 2D-Aβ-WIB from blood plasma;

FIG. 3: shows a comparison of A beta-peptide ratios (a) 4/15 (Aβ(1-42)/Aβ(2-40)), (b) 4/14 (Aβ(1-42)/Aβ(2-42)) and (c) 4/5 (Aβ(1-42)/Aβ(1-40)) in the population of the overall study (n=42), with classification or grading into patients having early/incipient AD (eiAD; D_Code_{—}1=1) and a control (Con; D_Code_{—}1=0) according to the clinical gold standard (diagram in the form of fields (box plots);

FIG. 4: shows a comparison of the A beta-peptide ratios (a) 4/5 (Aβ(1-42)/Aβ(1-40)), (b) 4/14 (Aβ(1-42)/Aβ(2-42)), and 4/15 (Aβ(1-42)/Aβ(2-40)) for a population examined, which is divided or graded into a group having eiAD (NDD_Code=1, n=18) and a group Con (NDD_Code=0, n=24) according to the neurochemical gold standard (diagram in the form of fields (box plots);

FIG. 5: shows a comparative ROC analysis for the A beta-peptide ratios 4/5 (Aβ(1-4)/Aβ(1-40)), 4/14 (Aβ(1-42)/Aβ(2-42)), and 4/15 (Aβ(1-42)/Aβ(2-40)) in patients having early/incipient AD (eiAD, n=21, D_Code_{—}1=1) versus control (Con, n=21, D_Code_{—}1=0), which are divided or graded according to the clinical gold standard;

FIG. 6: shows a comparative ROC analysis for A beta-peptide ratios 4/5 (Aβ(1-42)/Aβ(1-40)), 4/14 (Aβ(1-42)/Aβ(2-42)), and 4/15 (Aβ(1-42)/Aβ(2-40)) in patients having early/incipient AD (eiAD, n=18, NDD_Code=1) versus control (Con, n=24, NDD_Code=0), divided or determined according to the neurochemical gold standard;

FIG. 7: shows a comparative analysis based on a diagram in the form of fields (box plot) with a non-parametric evaluation of significance or meaningfulness (Kruskal-Wallis-H) for (a) Aβ peptide ratios 4/5 (Aβ(1-42)/Aβ(1-40)), (b) 4/14 (Aβ(1-42)/Aβ(2-42)) and (c) 4/15 (Aβ(1-42)/Aβ(2-40)) for patients where there is a CSF dementia biomarker and a statement regarding performance in the context of a cognitive test based on an MMSE (n=38);

FIG. 7a: shows the specific A beta-peptide ratio 4/5 (Aβ(1-42)/Aβ(1-40)); log values; significance value: p=0.132;

FIG. 7b: shows the specific A beta-peptide ratio 4/14 (Aβ(1-42)/Aβ(2-42)); log values; significance value: p=0.027;

FIG. 7c: shows the specific A beta-peptide ratio 4/15 (Aβ(1-42)/Aβ(2-40)); log values; significance value: p=0.013;

FIG. 8a: shows a corresponding scatterplot where the cut-off line y=−0.8737 originates from the ROC analysis for log 4vs5, NDD_Code=1 (cf. FIG. 6);

FIG. 8b: shows a scatterplot where the cut-off line y=1.0296 originates from the ROC analysis for log 4vs14, NDD_Code=1 (cf. FIG. 6);

FIG. 8c: shows a scatterplot where the cut-off line y=0.0938 originates from an ROC analysis for log 4vs15, NDD_Code=1 (cf. FIG. 6);

FIG. 9a: shows a scatterplot where the cut-off line y=−0.8737 originates from the ROC analysis for log 4vs5, NDD Code=1 (cf. FIG. 6);

FIG. 9b: shows a scatterplot where the cut-off line y=1.0296 originates from the ROC analysis for log 4vs14, NDD_Code=1 (cf. FIG. 6); and

FIG. 9c: shows a scatterplot where the cut-off line y=0.0938 originates from the ROC analysis for log 4vs15, NDD_Code=1 (cf. FIG. 6).

### DETAILED DESCRIPTION

It will be appreciated that particular configurations, embodiments or the like which are described only in connection with one aspect of the present invention also apply correspondingly hereinafter to the other aspects of the present invention, without requiring explicit mention.

Furthermore, all experimental investigations or studies or the like cited hereinafter can in principle be performed by methods or processes which are known per se to those skilled in the art or are standardized or are explicitly specified.

The present invention thus provides—in a first aspect of the present invention—for the use of at least one quantitative ratio (quotient) of two different amyloid beta-peptides (A-beta; Aβ) in a sample of a body fluid from a subject and/or patient for determination of his or her probability (risk) of contracting Alzheimer's disease (AD) and/or for determining (diagnosing) his or her suffering from a precursor of Alzheimer's disease, wherein the amyloid beta-peptides are selected from (a) Aβ(1-42), (b) Aβ(2-40) and (c) Aβ(2-42) and wherein the quantitative ratio of (a)/(b) or vice versa and/or of (a)/(c) or vice versa is formed.

In the context of the present invention, it is thus completely surprising that the objective can be achieved by the above-described inventive use of a ratio based on specific amyloid beta-peptides.

In this context, it is likewise completely unexpected that the ratio in question can be employed, and the presence of Alzheimer's disease to be reliably determined, or in order to reliably predictively determine the development of Alzheimer's disease.

The prior art does in principle disclose the connection between the occurrence of amyloid beta-peptides as such and Alzheimer's disease, and the general aspect of the use of amyloid beta-peptides is also considered in the prior art in relation to the diagnosis of Alzheimer's disease; however, until the time of the present invention, there has not been any kind of consideration in the prior art to consider such a specific ratio of amyloid beta-peptides underlying the present invention—and furthermore, more particularly, based on the use of a very specific sample, as will be detailed hereinafter—in order thus to enable a well-founded or reliable statement as to the presence of Alzheimer's disease or a reliable prognosis or prediction of the formation or development of Alzheimer's disease in a subject/patient.

In the context of the present invention, it is completely surprising that the specific ratio of particular amyloid beta-peptides is of high significance with regard to the presence of Alzheimer's disease, and the specific ratio of particular amyloid beta-peptides can thus be employed as a reliable indicator for the presence of Alzheimer's disease even at an early stage of the disease, for example, preclinical and early clinical forms of Alzheimer's disease.

The scope of the present invention involves a use or a method which can be performed ex vivo or in vitro on the basis of available samples of body fluids, especially blood-based samples, which provides good standardization with easy performability. Moreover, the inventive concept represents a distinct reduction in stress on the subject/patient, since the underlying sample, especially based on blood, can be obtained without any significant impairment or side effect.

A fundamental idea of the present invention is thus considered to be that of enabling a meaningful or reliable prognosis or prediction with regard to the development of Alzheimer's disease on the basis of the determination of the quantitative ratio of two specific amyloid beta-peptides. In the context of the present invention, however, not only is a meaningful prediction or prognosis possible with regard to the development or progress or Alzheimer's disease; instead, the present invention based on the analysis of the qualitative ratio of two specific amyloid beta-peptides in the available sample allows a meaningful determination or diagnosis with regard to any presence of Alzheimer's disease or Alzheimer's dementia—and already at a very early stage of the disease, in that this can already be determined in the preclinical or early clinical phase.

In this context, the applicant has found, completely surprisingly, that precisely the specific ratio of the amyloid beta-peptide Aβ(1-42), on the one hand, to the amyloid beta-peptide Aβ(2-40) or to the amyloid beta-peptide Aβ(2-42), on the other hand, from a sample of a body fluid, for example, blood, leads to particularly meaningful results with regard to the analysis of the presence of Alzheimer's disease or the prognosis of occurrence of Alzheimer's disease.

The principle underlying the present invention thus focuses on a neurochemical analysis of a sample of a body fluid, especially based on blood, with regard to the specific aforementioned amyloid beta-peptides in the sense of biomarkers specific to Alzheimer's disease or indicative of precursors thereof, in order in this way to enable the statements cited above with regard to Alzheimer's disease in a subject or patient.

In the context of the present invention, it is likewise possible to enable a differential diagnosis of Alzheimer's disease with exclusion of other dementia types since—in a completely unexpected manner—the applicant has found that the very specific ratios of the aforementioned amyloid beta-peptides in a defined sample are very specific with regard to Alzheimer's disease or its precursor or early forms. This is considered to be a further central advantage of the present invention.

According to the present invention, the aforementioned specific amyloid beta-peptides—namely Aβ(1-42), on the one hand, and Aβ(2-40) or Aβ(2-42), on the other hand—accordingly function as indicative biomarkers, in that they are specific for the presence of Alzheimer's disease in a patient or subject with regard to a concentration specific in each case and, resulting from this, with a specific ratio relative to one another. The amyloid beta-peptides in question and the specific concentrations or amounts thereof in a sample are thus correlated to Alzheimer's disease. The amyloid beta-peptides in question are thus, to some degree, indicators of pathological processes in the course of Alzheimer's disease.

The inventive concept is particularly meaningful firstly since it is based on the analysis of defined neurochemical biomarkers in a defined sample, these being directly correlated to Alzheimer's disease. Secondly, in addition, the inventive use and the inventive method, as will be described in detail hereinafter, can be performed in a much-simplified and inexpensive manner compared to the methods used in the prior art, such as psychometric methods, neuroimaging methods or the like.

On the basis of the use of specific biomarkers or bioindicators of the type mentioned—in addition to a statement as to whether Alzheimer's disease is present in a subject or patient or as to whether there is a certain probability with regard to the subject or patient that he or she will contract Alzheimer's disease—a further statement is also possible as to the extent to which any therapeutically active substances have a positive effect on the course of Alzheimer's disease, which means that the biomarkers can equally be employed as indicators for any medicament effects.

It is a feature of the present invention that, on the basis of the inventive concept, a rapid and meaningful analysis is possible with regard to Alzheimer's disease or with regard to the probability of occurrence of such a disease, which is additionally associated with only a low level of stress, if any, for the patient, more particularly with regard to the sample provision.

By virtue of the possibility, which arises from the present invention, of determining Alzheimer's disease as early as a preclinical or early clinical stage, or by virtue of the possibility of making a statement about any occurrence of Alzheimer's disease later in the life of the subject or patient, it is possible even at a very early stage, or for the purposes of a preventive approach, to react to the possible Alzheimer's disease, for example, with regard to a correspondingly optimized lifestyle of the subject or patient, or with regard to the targeted administration of medicaments which can, for example, improve the cognitive abilities of the subject or patient or delay the progression of the disease.

The underlying peptide Aβ(1-42) is especially an amyloid beta-peptide having a length of 42 amino acids, the first position of which or the amino-terminal end of which is formed by aspartate, whereas Aβ(2-40) and Aβ(2-42) are amyloid beta-peptides which have a length of 40 amino acids and 42 amino acids, respectively, each of which is truncated at its amino-terminal end. The specific amyloid beta-peptides in question are well known to the person skilled in the art as such, both in terms of their structure and in terms of their nomenclature, and so there is no need for any further details in this regard.

The term “quantitative ratio” or “quotient” as used in the context of the present invention focuses particularly on the relative concentration or relative amount of the aforementioned amyloid beta-peptides with respect to one another, and hence on a relative quantification of the amyloid beta-peptides in discussion. The determination of the respective concentration or amount and of the resulting ratios of the corresponding amyloid beta-peptides can be determined or found on the basis of analysis methods well known per se to those skilled in the art. A method in accordance with the present invention for determination of the respective ratios based on immunoprecipitation with a subsequent protein separation and detection method will be cited in detail hereinafter.

In the context of the present invention, the subject or patient may generally be a human or animal life form, more particularly a mammal. More particularly, the subject or patient is a human being. With regard to the sample or body fluid from the subject or patient, this may, in a nonrestrictive manner, comprise fluids which originate from or are formed by the subject's body. The sample or body fluid may especially be blood, plasma, lymph, urine and/or cerebrospinal fluid. As stated in more detail hereinafter, in the context of the present invention, the use of a body fluid based on blood, for example blood plasma or blood serum, is particularly advantageous especially since it is possible on this basis—in a completely surprising manner—to achieve particularly meaningful results, and the sampling is additionally simplified.

With regard, moreover, to the subject or patient as such, from which a sample of a body fluid is to be analyzed in the context of the present invention on the basis of the use or the method according to the present invention, the subject or patient may have symptoms of a deterioration in brain performance, particularly to a slight extent, for example, a deterioration in memory, particularly to a slight extent. With regard to the subject or patient, moreover, the subject or patient may especially have symptoms of mild cognitive impairment (MCI).

Mild cognitive impairment (MCI) may especially be an incipient dementia or an isolated impairment of memory. In general, the subjects or patients which are considered or examined further in the context of the inventive use or of the inventive method have cognitive impairments or weaknesses which are more marked than in individuals of comparable age and comparable constitution. More particularly, mild cognitive impairment (MCI) is characterized in that the symptoms are not (as yet) so marked that they lead to a lasting impairment in the daily activity of the subject or patient.

The inventive use or the inventive method is also especially of great interest in relation to patients or subjects with mild cognitive impairment (MCI), since people with mild cognitive impairment generally have an elevated probability of later contracting Alzheimer's disease. Thus, studies show that 10% to 15% of people affected by mild cognitive impairment contract Alzheimer's disease every year.

The present invention, however, is generally not restricted to subjects or patients with mild cognitive impairment (MCI), for which diagnostic clarification is possible, using the inventive use or inventive method, as to whether Alzheimer's disease is present and as to whether there is a risk of later development of Alzheimer's disease. Instead, it is also possible to examine those subjects or patients who do not have specific symptoms of a cognitive impairment or—more particularly with regard to a differential diagnosis—also those patients or subjects where there is at least the suspicion of a dementia other than Alzheimer's disease.

With regard to the precursor of Alzheimer's disease which can be determined or diagnosed on the basis of the present invention, this is especially a preclinical or subclinical stage of Alzheimer's disease. The preclinical precursor of Alzheimer's disease may especially be incipient or prodromal Alzheimer's disease, which is also referred to synonymously as incipient AD. In other words, in the context of the present invention, the precursor of Alzheimer's disease may be incipient or probable Alzheimer's disease. More particularly, the incipient Alzheimer's disease may be a precursor of Alzheimer's disease which cannot yet be diagnosed as an Alzheimer's disease at this disease stage with diagnostic methods established in the prior art.

According to the present invention, the precursor of Alzheimer's disease which can be detected on the basis of the inventive use or the inventive method may also be a clinical stage of Alzheimer's disease. More particularly, the precursor of Alzheimer's disease may also be early Alzheimer's disease, which is also referred to synonymously as early AD. More particularly, the early Alzheimer's disease may be an early stage Alzheimer's disease already clinically diagnosable on the basis of the methods known in the prior art.

More particularly, the precursor of Alzheimer's disease may be such a stage of Alzheimer's disease with advanced cognitive deficits, but where the cognitive deficits have not yet advanced to such an extent that these lead to a serious impairment in the daily life of the subject or patient.

According to the present invention, more particularly taking account of the above remarks regarding the term “Alzheimer's dementia”, it is thus possible on the basis of the inventive concept to enable a statement regarding the presence or regarding the future development of Alzheimer's dementia in a subject or patient, more particularly on the basis of the corresponding preclinical and/or clinical phases of Alzheimer's dementia on the basis of and/or as a consequence of an underlying Alzheimer's disease.

On this basis, the inventive use or the inventive method can thus be performed against the background of initiating measures even at an early stage which can delay or attenuate the progression of Alzheimer's disease, in order thus to maintain the quality of life of the patient or subject for as long as possible.

The ratio of the amyloid beta-peptides in question can, for example, be an amount- or concentration-based ratio.

Particularly good results with regard to the identification of Alzheimer's disease or with regard to the determination of the risk of contracting Alzheimer's disease can be achieved in accordance with the present invention when the ratio of (a) Aβ(1-42)/(b) Aβ(2-40) [i.e., Aβ(1-42)/Aβ(2-40) ratio or (a)/(b) ratio] and/or the ratio of (a) Aβ(1-42)/(c) Aβ(2-42) [i.e., Aβ(1-42)/Aβ(2-42) ratio or (a)/(c) ratio], for example, the ratio of (a) Aβ(1-42)/(b) Aβ(2-40) [i.e., Aβ(1-42)/Aβ(2-42) ratio or (a)/(c) ratio], is formed.

In an embodiment of the present invention, blood from the subject or patient can, for example, be used as the sample for determining the quantitative ratio of the amyloid beta-peptides in question. In this context, it is advantageous when, in the context of the present invention, blood plasma (plasma) or blood serum (serum), for example, blood plasma (plasma), from the subject or patient is used as the sample.

In this context, it is namely completely surprising that a significant change in the ratios in question for the particular amyloid beta-peptides correlated to Alzheimer's disease (and especially also with regard to early stages of the disease) is present in the blood from the subject or patient and especially in the plasma or serum.

In this context, the present invention is thus based on the completely surprising finding by the applicant that—without wishing to make any restriction to this theory—in the course of Alzheimer's disease—and also even at early stages of the disease—there is to some degree a holistic or overriding pathological “shift” in the overall organism or body, which is also associated with a significant change in the respective concentrations of the amyloid beta-peptides in question in the blood.

The fact that blood or blood plasma and/or blood serum of all substances can be employed for diagnosis of the presence of Alzheimer's disease is all the more surprising in that it has been assumed crucially to date that amyloid beta-peptides as such are not influenced in the blood, but primarily in the brain tissue itself or in the liquids connected to or associated with the brain, such as cerebrospinal fluid, in the course of the disease, and hence blood, to some degree, being a peripheral body fluid which is isolated from the brain itself, has not been cited as a crucial indicator in the prior art with regard to changes in the amyloid beta-peptides.

The completely surprising suitability of the use of blood or blood plasma and/or blood serum as the sample to be analyzed is also based on the completely surprising finding by the applicant that, more particularly also at the early stage of Alzheimer's disease, there is a fundamental “shift” in particular physiological parameters in the body of the person affected by the disease, and this is also reflected in a specific manner in the blood of the person affected, particularly with regard to the ratios in question for the aforementioned specific amyloid beta-peptides.

In this context, it is likewise completely surprising that especially the specific amyloid beta-peptide Aβ(2-40) can be employed, in that it cannot be detected in such a way in many other tissues or body constituents.

In this light too, it is completely unexpected in the context of the present invention that a specific ratio of amyloid beta-peptides, namely of Aβ(1-42), on the one hand, and Aβ(2-40) or Aβ(2-42), on the other hand, in the blood or in the blood plasma and/or blood serum can be employed in order to enable a reliable statement with regard to the determination of Alzheimer's disease or with regard to the determination of the probability of contracting such a disease.

It is additionally advantageous that blood can be made available in a simple manner, and the taking of blood is pain-free and essentially without risk to the patient or subject. This is considered to be a further advantage of the present invention.

In this embodiment of the present invention, more particularly, a neurochemical analysis or determination which is blood-based or based on blood tests for identification of dementia is thus provided, this being usable, for example, in the context of a corresponding diagnostic approach (blood based neurochemical dementia diagnostics (blood-NDD)).

As a result, it is a feature of the inventive concept that, firstly, a concept which is meaningful with regard to the determination of Alzheimer's disease—in spite of the generally low concentration of the amyloid beta-peptides to be analyzed in the sample—and, secondly, a concept which is gentle on the patient is provided, and this is suitable for repeated use, for example for the purpose of a follow-up check or the like.

With regard to the performance of the analysis of the sample to determine the relevant ratios, it is possible in this regard to use a number of purification and analysis methods known in the prior art.

Particularly good results are obtained when the amyloid beta-peptides, especially (a) Aβ(1-42), on the one hand, and (b) Aβ(2-40) and/or (c) Aβ(2-42), on the other hand, are removed or isolated from the sample, effectively in the manner of an upstream purification or separation step, especially by means of immunoprecipitation. The purification can be conducted in such a way that a collection of different types of amyloid beta-peptides, including (a) Aβ(1-42) and (b) Aβ(2-40) and/or (c) Aβ(2-42), is effectively removed from the underlying sample. This increases analysis accuracy.

In this context, it is especially possible, in the course of immunoprecipitation, to use at least one ligand which is specific to or binds to amyloid beta-peptides, for example, at least one antibody.

In this regard, the at least one ligand, for example, the at least one antibody, may be specific to amyloid beta-peptides of the (a) Aβ(1-y) and/or Aβ(2-y) type, where y is an integer from 37 to 43. In this context, the variable y characterizes particularly the length of the peptide in relation to the carboxy-terminal end thereof.

The ligand or the antibody in accordance with the present invention can, for example, be specific to (a) Aβ(1-42) and/or (b) Aβ(2-40) and/or (c) Aβ(2-42).

Moreover, the at least one ligand, for example, antibody, may be specific to the amino-terminal end of (a) Aβ(1-42) and/or (b) Aβ(2-40) and/or (c) Aβ(2-42).

More particularly, it is possible in accordance with the present invention to use a ligand or antibody which at least essentially has identical sensitivity or specificity at least for the specific amyloid beta-peptides Aβ(1-42), Aβ(2-40) and Aβ(2-42) in question, which is associated with the advantage that it is only necessary to use a single ligand or antibody type in the course of the purification step.

Furthermore, however, it is also possible to use several different types of ligands or antibodies with respective specificity for the amyloid beta-peptide to be isolated or to be analyzed.

For example, it is possible in accordance with the present invention to use the antibody of the 1E8 type. The antibody is especially sensitive to amyloid beta-peptides which have, with regard to their amino acid sequence, aspartate at position 1, such as (a) Aβ(1-42), or alanine at position 2, such as (b) Aβ(2-40) or (c) Aβ(2-42). The antibody of the 1E8 type can be purchased, for example, commercially from Bayer Schering Pharma AG, Berlin, Germany. An antibody in accordance with the present invention is described, more particularly, in EP 1 270 592 A1, the entire disclosure-content of which is hereby incorporated in full by reference.

With regard to the analysis of the sample in question, moreover, more particularly after the isolation or separation or purification of the amyloid beta-peptides to be determined which is conducted beforehand, the detection, especially the determination of the amount or content and/or concentration, of the respective amyloid beta-peptides, especially of (a) Aβ(1-42), on the one hand, and (b) Aβ(2-40) and/or (c) Aβ(2-42), on the other hand, can be performed on the basis of protein separation methods or on the basis of immunodetective protein detection methods, especially quantitative immunodetective protein detection methods, for example, on the basis of a combination of protein separation methods and immunodetective protein detection methods, especially quantitative immunodetective protein detection methods. It is especially possible in accordance with the present invention to proceed in such a way that the fraction comprising the various amyloid beta-peptides obtained in the course of the above-described purification method is correspondingly processed further or subjected to a further analysis.

In this context, the protein separation or protein detection method used may, for example, be a gel electrophoresis method, especially a two-dimensional gel electrophoresis, for example, a urea-based two-dimensional gel electrophoresis.

In addition, the protein separation method or protein detection method used may be a Western blot method.

Particularly advantageous results are achieved when the detection of the specific amyloid beta-peptides in question is performed on the basis of a combination of a two-dimensional gel electrophoresis, especially a urea-based two-dimensional gel electrophoresis, with a downstream Western blot method (2D-Aβ-WIB).

With regard to the protein separation or protein detection method used in the context of the present invention, moreover, it is also possible in the course of the immunodetective protein detection method, especially of the qualitative immunodetective protein detection method, for example, in the Western blot method, to use at least one ligand, for example, antibody, for labeling of the amyloid beta-peptides, especially in the manner of a primary ligand or primary antibody which binds directly to or interacts directly with the amyloid beta-peptides in question in of a first labeling step. In this context, the at least one ligand, for example, antibody, should be specific to amyloid beta-peptides of the Aβ(1-y) and Aβ(2-y) form, where y is an integer from 37 to 43. In addition, the ligand or antibody used in the course of the immunodetective protein detection method, especially the quantitative immunodetective protein detection method, should be specific to (a) Aβ(1-42) and/or (b) Aβ(2-40) and/or (c) Aβ(2-42). In addition, the at least one ligand, for example, antibody, should be specific to the amino-terminal end of (a) Aβ(1-42) or (b) Aβ(2-40) and/or (c) Aβ(2-42).

It is also the case for the immunodetective protein detection method, especially the quantitative immunodetective protein detection method, that it is possible to use either a ligand type or antibody type which has at least essentially identical sensitivity with respect to the aforementioned amyloid beta-peptides. For example, it is possible to use the above-described antibody of the 1E8 type. In addition, it is also possible to use various types of ligands or antibodies each with specific sensitivity for individual amyloid beta-peptides.

The specific detection can be made via the use of corresponding secondary ligands or secondary antibodies well known per se to those skilled in the art, to which, for example, enzymes or dyes, especially fluorescent dyes, can be coupled for the purposes of detection, the resulting conjugate being capable of delivering a detectable and evaluable measurement signal.

An identification or assignment of the specific amyloid beta-peptides can be made, for example, with reference to reference traces. The underlying principle is well known to those skilled in the art.

For further details of the methods for analysis of the sample in question which are usable in the context of the present invention, it is especially possible to refer to the scientific publication by Maler, J. M.: “Urea based two-dimensional electrophoresis of beta-amyloid peptides in human plasma: Evidence for novel Aβ species”, Proteomics, 2007, 7, 3815-3820, the entire disclosure-content of which is hereby fully incorporated by reference.

On the basis of the combination cited above for quantitative determination of the amyloid beta-peptides in question in the sample, based on an upstream immunoprecipitation and a downstream, especially quantitative, immunodetective protein detection method, a high accuracy with regard to the quantitative determination of the respective amyloid beta-peptides is ensured. Due to the method regime used in the context of the present invention for quantitative determination of the amyloid beta-proteins in the sample, it is possible to detect or record extremely low concentration of the peptides in question, and it is even possible in this regard to detect amounts or contents in the attomolar range.

With regard to the evaluation or assignment of the specific ratios of the amyloid beta-peptides in question found in the course of analysis of the sample with respect to the determination of the probability (risk) of the subject or patient contracting Alzheimer's disease, or with respect to the determination of his or her suffering from a precursor of Alzheimer's disease, it is advantageously possible in the context of the present invention to proceed in such a way that the quantitative ratio of two different amyloid beta-peptides, especially as defined above, is correlated or compared with corresponding reference ratios, or assigned to these reference ratios.

In other words, the assignment of the ratios of amyloid beta-peptides obtained from the analyzed sample can be made on the basis of a comparison with a reference system or a reference value or a reference value range, the corresponding reference parameters, as will be described below, being obtained in the form of statistical values, for example using a group of subjects or patients which serves as a reference.

This can be done by, for example, by first examining a group of reference subjects for the presence of cognitive deterioration or of precursors of Alzheimer's disease, for example on the basis of diagnostic methods known in the prior art, such as psychometric, neurochemical and/or neuroimaging methods, optionally with retrospective inclusion of subsequent actual development of Alzheimer's disease, and using this as a basis to conduct phenotyping with regard to the diagnostic finding or classification into particular reference groups with the particular clinical condition or the particular diagnosis, and, in these reference groups determined or formed beforehand, likewise analyzing the specific ratios of amyloid beta-peptides in a sample of the same kind and especially summarizing them statistically.

The assignment of the subject or patient to be examined is then made on the basis of a correlation or a comparison of the specific ratios found in the subject or patient in question with the ratios of the same kind found in the reference groups in question.

For instance, the procedure in the context of the present invention may be to use the comparison or the correlation of the quantitative ratio with the corresponding reference ratios to determine the probability of contracting Alzheimer's disease, and/or to determine or diagnose the presence of suffering from a precursor of Alzheimer's disease.

In this context, the term “correspondingly” as used in the context of the ratios to be compared for a subject or patient to be examined, on the one hand, and a reference group, on the other hand, relates especially to ratios which are the same or comparable in nature, i.e., especially to corresponding ratios of identical amyloid beta-peptides. For example, and in a nonrestrictive manner, the ratio of (a) Aβ(1-42)/(b) Aβ(2-40) for a subject can be compared with the correlating determined ratio (a) Aβ(1-42)/(b) Aβ(2-40) for the reference group. The ratio determined for the reference group is especially a statistical (mean) value or a value range for a group of patients belonging to this reference group.

The reference ratios determined are thus especially values or value ranges determined in relation to a group of reference subjects or reference patients in the sense of a statistical ratio based on numerous subjects, which may, for example, have been established beforehand. In this context, the values or value ranges ranges determined are especially statistically determined value ranges or statistical mean values with a corresponding standard deviation, which may be the basis for the assignment.

In the context of the present invention, it can be determined on the basis of, or to use as the basis, the quantitative reference ratios based on a reference group of subjects and/or a reference group of patients.

In this context, the reference subjects of the reference group of subjects or the reference patients of the reference group of patients may, more particularly for the purpose of a biostatistical evaluation, be examined for the presence or absence of a mild cognitive impairment (MCI) and/or of a preclinical precursor of Alzheimer's disease, especially incipient or prodromal Alzheimer's disease (incipient AD), and/or of a clinical precursor of Alzheimer's disease, especially early Alzheimer's disease (early AD).

In addition, in the context of the present invention, the examination or evaluation may be conducted on the basis of examination methods selected from the group of neurochemical methods, neuroimaging methods, psychometric methods and combinations of at least two of the aforementioned methods, for example, on the basis of a combination of all of the aforementioned methods. In this way, to some extent, an assignment of the corresponding reference subjects or reference patients with regard to their clinical condition is enabled.

In addition, the examination or evaluation with regard to the reference group of subjects or the reference group of patients can, for example, be performed on the basis of at least one gold standard (GS), especially selected from the group of clinical gold standards, neurochemical gold standards, psychometric gold standards and combinations of at least two of the aforementioned gold standards, for example, on the basis of a combination of all of the aforementioned gold standards.

The term “gold standard” (also referred to synonymously as GS) as used in the context of the present invention relates especially to an (examination) standard which is generally acknowledged at the particular examination time and which leads to particularly meaningful results with regard to the diagnosis of a disease in the context of “best practice”. The gold standard thus generally constitutes the actual or crucial standard. The gold standards used in the context of the present invention with regard to the analysis of the reference subjects or reference patients are sufficiently well known to the person skilled in the art as such. More particularly, the examination or evaluation with regard to the reference values can be conducted on the basis of gold standards which comprise psychometric, neurochemical or neuroimaging methods or parameters.

For example, the examination or evaluation can be conducted on the basis of a neurochemical method, the neurochemical method comprising the determination of parameters of cerebrospinal fluid (CSF); more particularly, the ratio of Aβ(x-42)/Aβ(x-40) [AβratioTGC], where x may independently assume the value of 1 or 2, and/or the content of Aβ(1-42) [Aβ142Inn] and/or the total content of tau and/or the content of phospho-tau 181 can be determined.

In addition, in the context of the classification of the reference subjects or reference patients, it is also possible to use neuroimaging methods, the neuroimaging methods comprising especially brain atrophy examinations, for example, based on imaging methods, for example, SPECT (Single Photon Emission Computed Tomography) and/or MRI (Magnet Resonance Imaging).

In addition, the psychometric method usable in accordance with the present invention may include studies based on MMSE (Mini-Mental State Examination).

More particularly, into the examination or evaluation, the later occurrence of Alzheimer's disease can be incorporated into the diagnosis at the examination time of the respective reference subject or reference patient, especially with regard to the assessment of the presence of a preclinical precursor of Alzheimer's disease at the examination time, if this cannot be determined sufficiently using the aforementioned diagnostic methods.

On the basis of the examination or diagnosis conducted in the reference patients or reference subjects, it is thus possible to assign the reference persons into particular classes with the corresponding clinical condition, for example, into a group comprising patients suffering from a cognitive impairment (MCI), and into at least one further group whose reference subjects or reference patients are suffering from a precursor of Alzheimer's disease. This can be conducted, for example, on the basis of an evaluation of the respective diagnostic methods, it being possible, for example, to establish graduated values or what are called cut-off values, which function to a certain degree as respective boundary values for the assignment to one of the aforementioned groups.

In the context of the present invention, more particularly, the reference subjects or reference patients may be divided into various reference groups as a function of the diagnosis made on the basis of the examination or evaluation.

In this context, the respective reference groups (A) and (B) can be formed from reference subjects or reference patients (A) not having diagnosed Alzheimer's disease or not having a diagnosed precursor of Alzheimer's disease and (B) having diagnosed Alzheimer's disease or having a diagnosed precursor of Alzheimer's disease, especially having a preclinical precursor of Alzheimer's disease, especially incipient or prodromal Alzheimer's disease (incipient AD), and/or having a clinical precursor of Alzheimer's disease, especially early Alzheimer's disease (early AD).

With regard to group (B) having diagnosed Alzheimer's disease or the diagnosed precursor of Alzheimer's disease, it is possible in this respect to make a further differentiation: for example, a reference group (B1) having a preclinical precursor of Alzheimer's disease, especially incipient or prodromal Alzheimer's disease (incipient AD), may form a further reference group (B2) having a clinical precursor of Alzheimer's disease, especially early Alzheimer's disease (early AD), and optionally a third reference group (B3) with diagnosed Alzheimer's disease at a far-advanced stage.

As stated above, quantitative reference ratios (reference quotients) of two different amyloid beta-peptides, especially as defined above, can be determined proceeding from a sample of a body fluid, especially as defined above, from each reference subject or reference patient belonging to a reference group and compiled for each of the respective reference groups. More particularly, the sample used for the purposes of correlation in relation to the reference patients or reference subjects in question from the respective reference groups is likewise blood or blood plasma and/or blood serum.

In this regard, in the context of the present invention, the procedure may especially be to correlate or compare the quantitative ratio of two different amyloid beta-peptides, especially as defined above, with the corresponding reference group, or to assign it to the corresponding reference group, in order in this way to determine the probability of the patient or subject contracting Alzheimer's disease, or in order in this way to determine the suffering of the patient or subject from a precursor of Alzheimer's disease.

In other words, in the context of the present invention, the procedure is especially to determine the quantitative ratio of (a) Aβ(1-42)/(b) Aβ(2-40) or the quantitative ratio of (a) Aβ(1-42)/(c) Aβ(2-42) in a blood sample or a sample of blood serum and/or blood plasma, and to correlate it to the respective corresponding, especially statistical, reference values—i.e., to the corresponding ratios of the same kind which have especially been obtained from a multitude of individuals from the respective reference group—from the blood samples or the samples of blood serum or blood plasma of the corresponding reference group, and to assign it to the corresponding reference group.

The assignment or correlation of the corresponding quantitative ratios of the respective amyloid beta-peptides is especially effected by assigning them to the range within a comparable order of magnitude which has been determined for the respective reference group. The ratios of subjects or patients, on the one hand, and reference groups, on the other hand, which are to be compared or correlated are thus, for example, corresponding ratios of the same size range, i.e., a ratio determined for a subject or patient can be assigned to that value range of the corresponding ratio from a reference group when the value determined for the subject or patient falls within the value range of the respective reference group.

On the basis of this correlation, it is possible, for example, to assign the subject or patient a relatively low or zero (i.e., an absent) probability of contracting Alzheimer's disease, or the subject and/or patient can be found not to be suffering from a preclinical stage of Alzheimer's disease, especially from incipient and/or prodromal Alzheimer's disease (incipient AD), and/or from a clinical precursor of Alzheimer's disease, especially early Alzheimer's disease (early AD), when the ratio determined for the subject or patient falls within the range of the corresponding reference ratios for reference group (A) not having diagnosed Alzheimer's disease and/or not having a diagnosed precursor of Alzheimer's disease.

In addition, in the context of the present invention, the subject or patient can be assigned an elevated probability of contracting Alzheimer's disease, or the subject or patient can be found to be suffering from a preclinical stage of Alzheimer's disease, especially from incipient and/or prodromal Alzheimer's disease (incipient AD), or from a clinical precursor of Alzheimer's disease, especially early Alzheimer's disease (early AD), when the ratio determined for the subject or patient falls within the range of the corresponding reference ratios for reference group (B) having diagnosed Alzheimer's disease or having a diagnosed precursor of Alzheimer's disease.

On the basis of the statements made above, it is likewise possible to conduct a further assignment or sub-differentiation when the aforementioned group (B), as described above, is divided into further subgroups (B1) and (B2) and optionally (B3), i.e., into a first subgroup (B1) having a preclinical stage of Alzheimer's disease, especially having incipient or prodromal Alzheimer's disease (incipient AD), and into a second subgroup (B2) having a clinical precursor of Alzheimer's disease, especially having early Alzheimer's disease (early AD).

In general, in accordance with the present invention, the ratio determined for the subject or patient can be assigned to one of the above-described reference groups (i.e., especially to reference group (A), (B) or (B1), (B2) or optionally (B3)) when the ratio determined for the subject or patient differs by at most 40%, especially at most 30%, for example, at most 25%, for example, at most 20%, for example, at most 10%, from the statistical mean of the reference ratio of the respective reference group, based on the aforementioned mean. Equally, as stated above, the ratio determined for the subject or patient can be assigned to one of the above-described reference groups when the ratio determined for the subject or patient is within the range of magnitude of the value range determined statistically for the particular reference group, or within the range of the calculated standard deviation of the statistical mean determined for the particular reference group.

On the basis of the above-described assignment, in accordance with the present invention, a statement is thus possible with regard to the clinical condition or the predictive course of the disease in the respective subject or patient.

More particularly, it is also possible in the context of the present invention to assign the subject and/or patient an elevated probability of contracting Alzheimer's disease, or to find the subject and/or patient to be suffering from a preclinical stage of Alzheimer's disease, especially from incipient and/or prodromal Alzheimer's disease (incipient AD), and/or from a clinical precursor of Alzheimer's disease, especially early Alzheimer's disease (early AD), when the amount and/or concentration of (b) Aβ(2-40) and/or (c) Aβ(2-42), especially of (b) Aβ(2-40), is elevated compared to the corresponding amounts and/or concentrations from the reference group not having diagnosed Alzheimer's disease and/or not having a diagnosed precursor of Alzheimer's disease, and consequently the ratio (a) Aβ(1-42)/(b) Aβ(2-40) or (a) Aβ(1-42)/(c) Aβ(2-42) is at the same time reduced.

In other words, in the event of an increase in the aforementioned individual values for the amyloid beta-peptides in question compared to the corresponding values for the reference group, and in the event of a consequently reduced ratio as defined above, there is an increased risk of contracting Alzheimer's disease or having such a disease, especially with regard to the aforementioned preclinical and clinical precursors. In this embodiment of the present invention, it is thus effectively possible to focus on the respective details of the amyloid beta-peptides in question.

This is because the applicant has found in this context that, surprisingly, especially the amount or concentration of amino-terminally truncated amyloid beta-peptides of the 2-y type, such as of the amyloid beta-peptides Aβ(2-40) and Aβ(2-42) in question, is elevated especially in the blood of those patients having an early precursor of Alzheimer's disease, especially a preclinical or clinical precursor of Alzheimer's disease, in connection with a mild cognitive impairment (MCI).

In addition, it is possible in the context of the inventive use that the subject and/or patient is assigned an elevated probability of contracting Alzheimer's disease, and/or the subject and/or patient is acknowledged to be suffering from a preclinical stage of Alzheimer's disease, especially from an incipient and/or prodromal Alzheimer's disease (incipient AD), and/or from a clinical precursor of Alzheimer's disease, especially early Alzheimer's disease (early AD), when the relative ratio of Aβ(1-42)/Aβ(2-y) is reduced compared to the corresponding reference ratios for the reference group not having diagnosed Alzheimer's disease and/or not having a diagnosed precursor of Alzheimer's disease, where y is an integer from 37 to 43, especially 40 or 42, and/or when the relative ratio of (a) Aβ(1-42)/(b) Aβ(2-40) and/or (a) Aβ(1-42)/(c) Aβ(2-42), especially (a) Aβ(1-42)/(b) Aβ(2-40), is reduced compared to the corresponding reference ratios for the reference group not having diagnosed Alzheimer's disease and/or not having a diagnosed precursor of Alzheimer's disease.

In this embodiment of the present invention, the emphasis is thus on an analysis of the aforementioned specific ratios as such from the respective amyloid beta-peptides. This is because, in addition to the statements made above for the individual values, the applicant has likewise found that, completely surprisingly, a preclinical or clinical Alzheimer's disease, especially incipient or prodromal Alzheimer's disease, is associated with a significant decrease in the aforementioned ratios, especially in blood.

This connection, shown for the first time by the applicant, is also completely surprising against the background that, in general, there is no direct correlation between the corresponding dementia biomarkers in other constituents of the body, such as cerebrospinal fluid, with the specific ratios of amyloid beta-peptides of the aforementioned type found particularly in the blood.

Particularly through the selective combination of specific diagnostic methods in the context of the classification of the reference groups, it has been possible in the context of the present invention to show the correlation or the connection of the aforementioned amyloid beta-peptides and the corresponding specific ratios in the blood, especially with specific preliminary forms of Alzheimer's disease, such that this connection, in the context of the present invention, is the basis of a meaningful test which is to be conducted using standardized methods and is based on a blood analysis for the presence of Alzheimer's disease or a corresponding precursor.

The applicability of the inventive use of specific ratios of amyloid beta-peptides, especially on the basis of a blood sample, is all the more surprising in that it is common knowledge that there is specifically no direct connection between the specific dementia biomarkers, for example, in cerebrospinal fluid, to those in blood. In this regard, it is also notable that, in relation to blood as such, numerous peripheral cell systems or cell populations, for example liver cells, blood platelets, monocytes or the like, can influence the ratios in question, and so it is generally actually impossible to conclude from the specific formation of A beta-peptides in the cerebrospinal fluid that these ratios are present in the blood, or vice versa.

Therefore, it was not foreseeable in accordance with the present invention that a durable and relatively reliable statement regarding the presence of Alzheimer's precursors of the aforementioned type is possible on the basis of an analysis of a blood sample.

The inventive use can also be conducted in combination with further analysis methods, for example, in combination with a specific analysis of the ratio of Aβ(1-42)/Aβ(1-40), more particularly in the cerebrospinal fluid, and this further improves the meaningfulness.

The present invention further provides—in a second aspect of the present invention—an inventive method for determining the probability (risk) of a subject and/or patient contracting Alzheimer's disease (AD), wherein at least one quantitative ratio (quotient) of two different amyloid beta-peptides (A-beta; Aβ) in a sample of a body fluid from the subject and/or patient is determined, wherein the amyloid beta-peptides are selected from (a) Aβ(1-42), (b) Aβ(2-40) and (c) Aβ(2-42) and wherein the quantitative ratio of (a)/(b) or vice versa and/or of (a)/(c) or vice versa is formed.

The present invention likewise relates—in a third aspect of the present invention—to an inventive method for determining the probability (risk) of a subject and/or patient contracting Alzheimer's disease (AD), especially as defined above,
(a) wherein at least one quantitative ratio (quotient) of two different amyloid beta-peptides (A-beta; Aβ) in a sample of a body fluid from the subject and/or patient is determined, wherein the amyloid beta-peptides are selected from (a) Aβ(1-42), (b) Aβ(2-40) and (c) Aβ(2-42) and wherein the quantitative ratio of (a)/(b) or vice versa and/or of (a)/(c) or vice versa is formed;
(b) wherein the ratio thus obtained is compared and/or correlated with corresponding reference ratios and/or wherein the ratio thus obtained is assigned to corresponding reference ratios; and
(c) wherein, on the basis of the comparison and/or the correlation and/or the assignment, the probability of the patient and/or subject contracting Alzheimer's disease is subsequently determined.

For further details, to avoid unnecessary repetition, reference may be made to the other aspects of the present invention, which apply correspondingly in relation to the inventive methods.

Finally, the present invention further provides—in a fourth aspect of the present invention—an inventive kit, especially for determining the probability (risk) of a patient and/or subject contracting Alzheimer's disease (AD) and/or especially for determining (diagnosing) his or her suffering from a precursor of Alzheimer's disease, wherein the kit comprises components and/or compositions for determining a quantitative ratio (quotient) of two different amyloid beta-peptides (A-beta; Aβ) in a sample of a body fluid from a subject and/or patient, wherein the components and/or compositions are selected such that they enable the quantitative determination of amyloid beta-peptides from the group of (a) Aβ(1-42), (b) Aβ(2-40) and (c) Aβ(2-42) for the purposes of determining the quantitative ratio of (a)/(b) or vice versa and/or of (a)/(c) or vice versa.

The components or compositions of the inventive kit may especially be compositions or components for purification, concentration, separation or the like of the amyloid beta-peptides to be examined. Also useful in this regard are compositions or components familiar per se to the person skilled in the art for protein separation, as used, for example, in the context of immunodetective methods or of separation methods known per se, such as gel electrophoresis methods.

More particularly, the compositions or components may, however, also be substances which label or interact with the amyloid beta-peptides to be analyzed, such as ligands or antibodies.

In this context, the inventive kit may comprise at least one ligand, for example, antibody, which is specific to and/or binds to amyloid beta-peptides. In this regard, the at least one ligand, for example, antibody, may be specific to amyloid beta-peptides of the Aβ(1-x) and Aβ(2-y) type, where y is an integer from 37 to 43.

In addition, the at least one ligand, for example, antibody, may be specific to (a) Aβ(1-42) and/or (b) Aβ(2-40) and/or (c) Aβ(2-42).

In addition, the at least one ligand, for example, antibody, may be specific to the amino-terminal end of (a) Aβ(1-42) and/or (b) Aβ(2-40) and/or (c) Aβ(2-42).

In relation to further details regarding the inventive kit, reference may be made to the above remarks regarding the inventive use and the inventive methods, which equally apply in a corresponding manner to the inventive kit.

Further advantages, features, properties and aspects of the present invention are evident from the remarks which follow, which describe the inventive concept in detail, along with the working examples which follow and the appended figures.

The focus of the present invention, with regard to sample analysis, is especially on a method or a process for amino terminal-selective immunoprecipitation with a subsequent attomolar-sensitive, especially urea-based, two-dimensional Western immunoblot (2D-Aβ-WIB), in order to examine the complex beta-amyloid peptide signature in the EDTA blood plasma of subjects (n=42) which are formed from cases with early or incipient Alzheimer's disease (eiAD, n=21) and control subjects (Con, n=21).

The present invention relates more particularly to the identification of particular A beta-peptide ratios (A beta-peptide quotients) in the blood, these showing highly significant differences between eiAD and Con patients.

More particularly, the formation of Aβ(2-y) species is elevated in relation to Aβ(1-42) in eiAD patients. Surprisingly, the effect mentioned is particularly marked at preclinical stages (incipient AD), for example, in patients with mild cognitive impairment (MCI) which have prodromal Alzheimer's disease according to the dementia biomarker pattern of their cerebrospinal fluids.

Consequently, it is possible in accordance with the present invention to use specific A beta-peptide ratios of Aβ(1-42) to particular Aβ(2-y) species, especially the Aβ(1-42)/Aβ(2-40) and Aβ(1-42)/Aβ(2-42) ratios, for neurochemical diagnosis of early and/or incipient Alzheimer's disease by analysis of body fluids, for example, blood (for example in combination with the Aβ(1-42)/Aβ(1-40) ratio). This enables, more particularly, a blood-based or blood test-based neurochemical dementia diagnosis of incipient (preclinical/prodromal) Alzheimer's disease (AD). The present invention thus also enables newly directed strategies for secondary preventive treatment, especially of high-risk patients.

In this context, as evidence of its invention, the applicant has conducted an amino terminal-selective immunoprecipitation with subsequent attomolar-sensitive urea-based two-dimensional Western immunoblot (2D-Aβ-WIB), in order to examine the complex beta-amyloid peptide signature in EDTA blood plasma of subjects (n=42) including cases of early or incipient Alzheimer's disease (eiAD, n=21) and a disease control group (Con, n=21).

The clinical phenotyping of the patients having Alzheimer's disease is supported by CSF-NDD and, in a majority of the cases, by neuroimaging methods (MRI/SPECT). For biostatistical evaluation, the patients are graded or classified in accordance with a clinical gold standard, a neurochemical gold standard and gold standards (GS) which combine psychometric (MMSE) and neurochemical parameters (CSF dementia biomarkers) and also neurochemical parameters (CSF dementia biomarkers) and neuroimaging process parameters (SPECT). Biomarker-promoted gold standards (neurochemical GS; combined neurochemical GS and SPECT GS) are used in order to achieve objective assignment of the patients, as a result of which cross-validation of the clinically supported phenotyping of the early or incipient Alzheimer's disease is achieved.

The 2D-Ab-WIB analysis of the blood signature of A beta-immunoreactive peptides shows a stable pattern of 18 fields (spots), which has been quantified with reference to a mixture of A beta-standard peptides. The biostatistical analysis shows that none of the individual A beta-immunoreactive fields showed a significant difference between the eiAD and Con patient groups. Only field 4, which corresponds to Aβ(1-42) shows a trend for a disease-specific difference. Specific A beta-peptide ratios show highly significant differences between eiAD and Con patients. This effect is determined primarily by the ratio of Aβ(1-42) (field 4) to other A beta-peptide species which are amino-terminally truncated at the first amino acid, in this case aspartate (Aβ(2-x) species). Consequently, the ratio of Aβ(1-42) to the sum of the Aβ species (2-42) (field 14), (2-40) (field 15), (2-39) (field 16), (2-38) (field 17) and (2-37) (field 18) distinguishes the eiAD and Con groups significantly from one another, whereas this is not the case for the ratios of the other Aβ(1-x) species (e.g. Aβ(1-40)) to the sum of Aβ(2-x) species or to individual Aβ(2-x) species.

In the group of the Aβ(1-x) peptides, only the ratio of Aβ(1-42) (field 4) to Aβ(1-40) (field 5) permits a statistically significant distinction of the eiAD and Con patient groups. A refined data analysis shows that the improved diagnostic performance of the ratio of Aβ(1-42) to the sum of the Aβ(2-x) species is explained primarily by the ratio of Aβ(1-42) to Aβ(2-40) (ratio of fields 4/15), followed by the ratio of Aβ(1-42) to Aβ(2-42) (ratio of fields 4/14). The diagnostic accuracy of the ratio of Aβ(1-42)/Aβ(2-40) (ratio of fields 4/15) is distinctly superior in the ratio Aβ(1-42)/Aβ(1-40) (fields 4/5), which is currently regarded as the gold standard for blood-based neurochemical diagnosis methods for recognition of dementia, especially of Alzheimer's disease.

Correlation analysis additionally permits the conclusion that the ratio of Aβ(1-42) to amino-terminally truncated Aβ(2-x) species, especially Aβ(1-42)/Aβ(2-40) (ratio of fields 4/15), is much more closely correlated with the CSF biomarkers for dementia than the Aβ(1-42)/Aβ(1-40) ratio. This relationship is at its strongest for the correlation of the Aβ(1-42)/Aβ(2-40) ratio (ratio of fields 4/15) to the total content of tau protein (total tau). In addition, a significant correlation of the Aβ(1-42)/Aβ(2-40) ratio (ratio of fields 4/15) is observed with routine brain perfusion SPECT analyses (^{99m}Tc SPECT), whereas no such correlation can be observed for the Aβ(1-42)/Aβ(1-40) ratio (ratio of fields 4/5). Furthermore, the Aβ(1-42)/Aβ(2-40) ratio (ratio of fields 4/15) is more closely correlated to the ApoE-e4 genotype, a proven risk factor for Alzheimer's disease, than the Aβ(1-42)/Aβ(1-40) ratio. Secondly, Aβ(1-42)/Aβ(2-40) (ratio of fields 4/15) is significantly more closely correlated to the age of the patients than the Aβ(1-42)/Aβ(1-40) ratio.

A significant correlation is also observed between the age and total tau, which is known from the literature. Since the eiAD and Con patient groups are not of the same age, it cannot be ruled out that some of the observations can be explained primarily by the age and not by disease-related differences. Age is, however, the most important known risk factor for Alzheimer's disease, and total tau has recently been identified as the highest-performance individual CSF biomarker for forecasting of incipient Alzheimer's disease in MCI patients. This effect was also independent of the age of the MCI patients. Furthermore, another current brain perfusion SPECT study shows that, among the CSF biomarkers for recognition of dementia (phospho-tau, total tau, Aβ(1-42)), total tau and phospho-tau, but not Aβ(1-42), have significant correlation with local disruption of perfusion (left parietal cortex) in the event of early Alzheimer's disease and MCI.

Surprisingly, the applicant can observe a noticeable decrease in the Aβ(1-42)/Aβ(2-40) ratio (ratio of fields 4/15) in the blood of MCI patients having prodromal Alzheimer's disease (incipient Alzheimer's disease), the Alzheimer's disease being indicated according to the CSF biomarker signature. This finding could not be observed for the Aβ(1-42)/Aβ(1-40) ratio (ratio of fields 4/5) in the blood. Indications from the biochemical analysis of beta-amyloid plaques from brain tissue in a wide variety of different disease stages of human Alzheimer's disease show clearly that the formation of amino-terminally truncated A beta-peptide species is a very early event in the molecular pathogenesis of Alzheimer's disease. Consequently, enhanced formation of Aβ(2-x) species in relation to Aβ(1-42) at preclinical stages (incipient Alzheimer's disease) may be particularly marked, for example, in MCI patients having prodromal MCI Alzheimer's disease.

In this connection, the behavior of the amount or the content of A beta-peptides in relation to blood or blood plasma and/or blood serum as the sample to be analyzed is such that the amounts or contents of A beta-peptides found therein do not have significant correlation with CSF A beta-peptides, i.e., the A beta-peptide blood profile cannot be considered to be a kind of “diluted” CSF profile. Furthermore, the amount or the content of A beta-peptide in the blood depends in a much more complex manner on factors other than the CSF pattern, since numerous peripheral cell systems or cell populations (liver cells, blood platelets, monocytes etc.) contribute to or influence the pattern. Accordingly, it is neither possible nor justified to transfer or to generalize findings or relationships from studies on A beta-peptides which have been identified as CSF biomarkers for recognition of dementia to the blood or to blood tests. For example—in contrast to blood—no indications can be found for comparably high concentrations of Aβ(2-40) (relative to Aβ(1-42) or to the total content of A beta-peptides) in CSF samples.

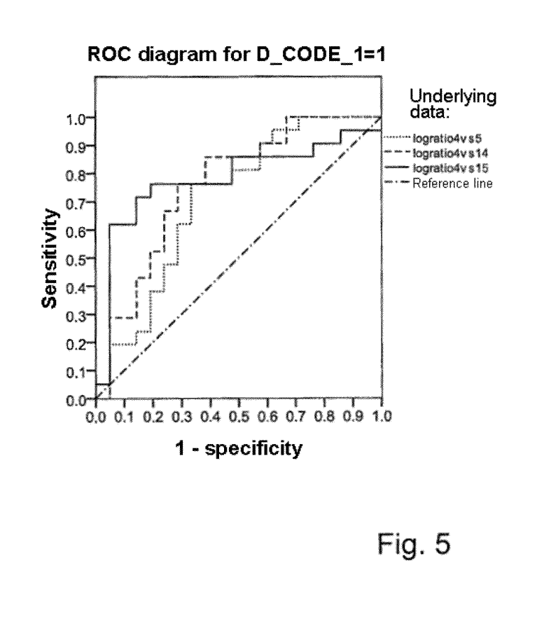

The comparative ROC analysis (FIG. 5) of the ratios of fields 4/5, 4/14 and 4/15 shows clearly that the combined use of the ratios of fields 4/5, 4/14 and 4/15 has an enhanced diagnostic value. In the case of moderate sensitivity (e.g. 70 to 80%), the ratio of fields 4/15 shows improved specificity, whereas, in the case of high sensitivity (90 to 100%), the ratios of fields 4/5 and 4/14 give better results. This becomes particularly clear when a logistic regression analysis (Statistical Package of Social Sciences, SPSS, Version 17) is employed (exact analysis not shown) in order to classify the patients by the clinical or neurochemical gold standard.

When all three ratios are employed, 81% or 85.7% of the patients are classified correctly by the clinical or neurochemical gold standard. If only the ratio of fields 4/5 is employed, only 66.7% or 61.9% of the patients are correctly classified by the clinical or neurochemical gold standard.

### 1. Methods

### 1.1 Patient Cohorts and Clinical Phenotyping

The complex beta-amyloid peptide signature in the EDTA blood plasmas of 42 subjects was examined. All patients were recruited in the psychiatry department of the University of Erlangen/Nuremburg, Germany. The study population was graded or divided into 21 patients having early or incipient Alzheimer's disease (eiAD; D_Code_{—}1=1) and 21 control patients (Con; D_Code_{—}1=0). The control cases included patients having heterogeneous neuropsychiatric disorders, including other dementias. CSF-based neurochemical diagnostic methods for recognition of dementias were available for all patients in the study. The clinical gold standard (clinical GS) for the phenotyping of the 42 patients, pre-analysis sample treatment and CSF-supported neurochemical diagnostic methods for recognition of dementias (CSF-NDD) were guided by the international diagnosis guidelines of the German Competence Network for Dementias (www.kompetenznet-demenzen.de; Kornhuber et al.: “Early and differential diagnosis of dementia and mild cognitive impairment: design and cohort baseline characteristics of the German Dementia Competence Network”, Dement. Geriatr. Cogn. Disord., 2009, 27, 404-417; Wiltfang et al.: “Consensus paper of the WFSBP Task Force on Biological Markers of Dementia: the role of CSF and blood analysis in the early and differential diagnosis of dementia”, World J. Biol. Psychiatry, 2005, 6, 69-84; Lewczuk et al.: “The German Competence Net Dementias: standard operating procedures for the neurochemical dementia diagnostics.”, J. Neural Transm., 2006, 113, 1075-80; Lewczuk and Wiltfang: “Neurochemical dementia diagnostics: State of the art and research perspectives.” Proteomics 2008, 8, 1292-301).

In addition, for further validation of the diagnosis of Alzheimer's disease, patients having early/incipient Alzheimer's disease (eiAD) included, for example, in the study were those where neuroimaging methods (SPECT and/or cMRI) supported the diagnosis of Alzheimer's disease. eiAD patients having a neuroimaging finding which indicated the presence of other dementias (for example vascular dementia or frontotemporal dementia) were not included in the study.

In order to enable a substantially standardized interpretation of the data from routine neuroimaging methods, the following division criteria were used:
1.5 tesla MRI or ^{99m}Tc SPECT_Score “3”: frontotemporoparietal atrophy, frontotemporoparietal perfusion deficit, condition was classified as typical of Alzheimer's disease, no significant lesions, no neoplasma;
score “2”: other brain atrophy, generalized atrophy, significant number of lesions, indications of neoplasma;
score “1”: other pathological findings;
score “0”: no pathological findings.

MRI and SPECT neuroimaging methods were available for 39 and 37 patients respectively. In addition, eiAD patients, for example, included in the study were those where the clinical diagnosis was supported by a CSF dementia biomarker pattern typical of Alzheimer's disease (lowered: Aβ(1-42), Aβ42/Aβ40 ratio; elevated: total tau, phospho-tau181). In contrast, control cases having CSF-NDD typical of Alzheimer's disease were excluded. For the patients, a risk of incipient Alzheimer's disease was found when they already showed the CSF dementia biomarker pattern indicating Alzheimer's disease and their cognitive deficit patterns indicated mild cognitive impairment.

In addition to the clinical gold standard, a neurochemical gold standard based on CSF biomarkers for recognition of dementias was employed. The neurochemical gold standard takes account of the quantitative information which is provided by all four CSF biomarkers for recognition of dementias (total tau, phospho-tau181, Aβ(1-42) and Aβ(1-40)). This information was used in order to obtain the NDD_Score (ordinal scale) and a derived dichotomic NDD_Code. The NDD_Code was combined with the results of the mini mental state examination test (MMSE) in order to obtain or to substantiate the neurochemical diagnostic methods for recognition of dementias and the cognition code (NDD_Cog-Code). Both codes enabled objective (NDD_Code; n=42) or substantially objective (NDD_Cog_Code; n=38) phenotyping of the patients, based on their molecular and/or cognitive biomarker information.

Since the qualitative evaluation of the CSF biomarkers was already used to support the clinical gold standard (see above), a high degree of agreement was achieved between the clinical and neurochemical gold standard. The definition of the NDD_Code and of the NDD_Cog_Code was explained in detail hereinafter. Finally, the NDD_Score (range: −1 to +2) and the SPECT_Score (range: 0 to 3) were combined in order to obtain the NDD_SPECT_Score (range: −1 to +5; n=37). The NDD_SPECT_Score corresponds to the sum of the NDD and SPECT scores.

### 1.2 Molecular Phenotyping and Neurochemical Gold Standard: Derivation of the NDD_Score

The inaccuracy of the clinical diagnosis of early Alzheimer's disease (AD) is significant. According to studies supported by autopsies, the accuracy even at moderate to far-advanced stages of Alzheimer's disease is at best 85 to 90%. Since the clinical syndrome at early stages of Alzheimer's disease is still very much more heterogeneous, the accuracy of clinical diagnosis in this case should not exceed 85%.

There is a weight of evidence from the meta analysis of several large international multicenter studies and from autopsy-supported studies that a specific biomarker signature for dementias in cerebrospinal fluid (CSF) strongly supports the correct diagnosis of early and even incipient (preclinical) Alzheimer's disease. This explains why CSF-supported neurochemical diagnostic methods for recognition of dementias (CSF-NDD) were incorporated in 2010 into the German neuropsychiatric S3 guidelines for the improved diagnosis of early dementias. The clinical relevance of CSF-NDD is indicated by a large amount of literature evidence (Hansson et al.: “Association between CSF biomarkers and incipient Alzheimer's disease in patients with mild cognitive impairment: a follow-up study.”, Lancet Neurol., 2006, 5, 228-234; Engelborghs et al.: “Neuropsychological and behavioural correlates of CSF biomarkers in dementia.”, Neurochem. Int., 2006, 48, 286-295; Engelborghs et al.: “No association of CSF biomarkers with APOEepsilon4, plaque and tangle burden in definite Alzheimer's disease.” Brain, 2007, 130, 2320-2326).

Since the patient cohort examined in the study described here consists principally of patients having early or incipient (preclinical/prodromal) Alzheimer's disease, the information from the CSF biomarker signature for dementias was used as a judgement basis for an objective diagnostic grading or division of the patient group (neurochemical gold standard), which was independent of the conventional clinical stages. This was achieved by the founding or establishment of a neurochemical dementia code (NDD_Code), which was in turn obtained from an averaged neurochemical diagnostic score for dementias (avNDD_Score).

The NDD score was based on the information from the following four biomarkers for dementias in the cerebrospinal fluid (CSF):
ratio of the A beta-peptides Aβ(x-42) to Aβ(x-40) (AβratioTGC). For the determination, two ELISA tests from THE GENETICS COMPANY, Zurich are used, which do not specifically measure the Aβ(1-42) and Aβ(1-40) peptides, but also the amino-terminally truncated species thereof;
Aβ(1-42) (Aβ142Inn): ELISA test from INNOGENETICS, Gent, specifically measures Aβ(1-42);
total tau (ELISA test from INNOGENETICS);
phospho-tau181 (ELISA test from INNOGENETICS).

CSF biomarker information for dementias was available for all patients, but not for all biomarkers for all patients (total tau: n=42, Aβ142Inn: n=42, phospho-tau: n=29, AβratioTGC: n=20).

Patients having a CSF biomarker concentration for dementias within the high normal range were assigned, in an arbitrary or freely selected manner, an NDD score of “−1”. In contrast, patients having pathological values for CSF dementia biomarkers close to the cut-off value, above the cut-off value and far above the cut-off value were assigned NDD scores of “0”, “1” and “2” respectively. Since not all four biomarkers were available for recognition of dementias for each individual patient, an averaged NDD score (avNDD_Score) was calculated, i.e., ΣScore/n. An avNDD_Score of −1 and +2 corresponds, respectively, to a minimum and maximum risk of incipient or obvious clinical Alzheimer's disease.

The ordinal avNDD_Score was used in order to obtain the dichotomic NDD_Code: an avNDD_Score of <1 corresponds to a low risk of incipient or obviously clinical Alzheimer's disease (NDD_Code=0), whereas an avNDD_Score of 1 corresponds to a high risk of incipient or obvious clinical Alzheimer's disease (NDD_Code=1).

The cut-off values which define the classification ranges of the NDD_Score were taken from the literature or derived from our own laboratory work (e.g. AβratioTGC): total tau=350 pg/ml; phospho-tau181=60 pg/ml; Aβ142Inn=530 pg/ml; AβratioTGC=0.09. The ranges of the concentrations for CSF biomarkers for recognition of dementias which define the different NDD_Score values are summarized in table 1.

TABLE 1
CSF dementia biomarker values which justify the NDD score

| Parameter: | Parameter range (pg/ml): | Score: |
|---|---|---|
| PhosphoTAU181 | 0-50 | −1 |
| PhosphoTAU181 | >50 and <60 | 0 |
| PhosphoTAU181 | ≧60 and <70 | 1 |
| PhosphoTAU181 | ≧70 | 2 |
| TotalTAU | 0-250 | −1 |
| TotalTAU | >250 and <350 | 0 |
| TotalTAU | ≧350 and <450 | 1 |
| TotalTAU | ≧450 | 2 |
| Aβ142Inn | 0-300 | 2 |
| Aβ142Inn | >300 and <530 | 1 |
| Aβ142Inn | ≧530 and <650 | 0 |
| Aβ142Inn | ≧650 | −1 |
| AβratioTGC | 0-0.08 | 2 |
| AβratioTGC | >0.08 and <0.10 | 1 |
| AβratioTGC | ≧0.10 and <0.13 | 0 |
| AβratioTGC | ≧0.13 | −1 |

According to the neurochemical gold standard, 18 patients were graded or classified as eiAD (NDD_Code=1) and 24 patients as Con (NDD_Code=0).

### 1.3 Combined Molecular and Cognition Phenotyping: the Neurochemical Dementia and Cognition Score

The score for neurochemical diagnostic methods in the case of dementias and cognition (NDD-Cog-Code) combines the CSF-supported neurochemical detection for incipient or obvious Alzheimer's disease—as expressed by the NDD_Code—with the determination of the cognition deficits—as expressed by the mini mental status test (MMSE). An MMSE score of less than 25 units indicates dementia, though a maximum score of more than 30 units does not rule out mild cognitive impairment since the MMSE is not sensitive enough to indicate subtle cognitive deficits. The parameter requirements which define the NDD-Cog_Code are summarized in table 2.

TABLE 2
Parameter settings for the NDD_Cog_Code

| | | | |
|---|---|---|---|
| Other dementias (oD); | MMSE < 25 | AvNDD_Score < 1.0 | NDD_Cog_Score = 3 |
| n = 3 | | | |
| Early Alzheimer's disease | MMSE < 25 | AvNDD_Score ≧ 1.0 | NDD_Cog_Score = 2 |
| (eAD); n = 15 | | | |
| Incipient Alzheimer's | MMSE 25-30 | AvNDD_Score ≧ 1.0 | NDD_Cog_Score = 1 |
| disease (iAD); n = 2 | | | |
| No Alzheimer's disease | MMSE 25-30 | AvNDD_Score < 1.0 | NDD_Cog_Score = 0 |
| (NoAD); n= 18 | | | |

### 1.4 Amino Terminal-Selective Immunoprecipitation and Attomolar-Sensitive Urea-Based Two-Dimensional Western Immunoblot (2D-Aβ-WIB)

In the course of the studies conducted, an amino terminal-selective immunoprecipitation and an attomolar-sensitive urea-based two-dimensional Western immunoblot (2D-Aβ-WIB) according to Maler et al., 2007 were used in order to study the complex beta-amyloid peptide signature in EDTA blood plasma. Individual peptide concentrations were quantified by Western immunoblots in relation to a mixture of synthetic A beta-peptides, which run as one-dimensional marker traces at the side of each two-dimensional Western immunoblot. A beta-immunoreactive fields which are separated by 2D-Aβ-WIB were identified by their two-dimensional migration characteristics compared to known synthetic A beta-peptides and by amino and carboxyl terminal-selective immunoprecipitation and subsequent 2D-Aβ-WIB. In addition, further molecular evidence from an independent study (Schieb H. et al., publication being prepared) was used, which was obtained from the analysis of brain samples from the transgenic mouse model of Alzheimer's disease (mouse model APP23), the brain homogenates being separated by 2D-Aβ-WIB and the fields subsequently being analyzed by mass spectrometry.

### 1.5 Biostatistical Data Analysis

Because of the distorted data distribution of several A beta-peptides from blood plasma which were separated by 2D-Aβ-WIB, the raw concentrations were logarithmically transformed. Accordingly, the biostatistical calculations (SPSS™) were conducted with the logarithmized data, including the calculation of the ratios (quotients).

Further advantages, features, properties and aspects of the present invention are evident from the appended figures.

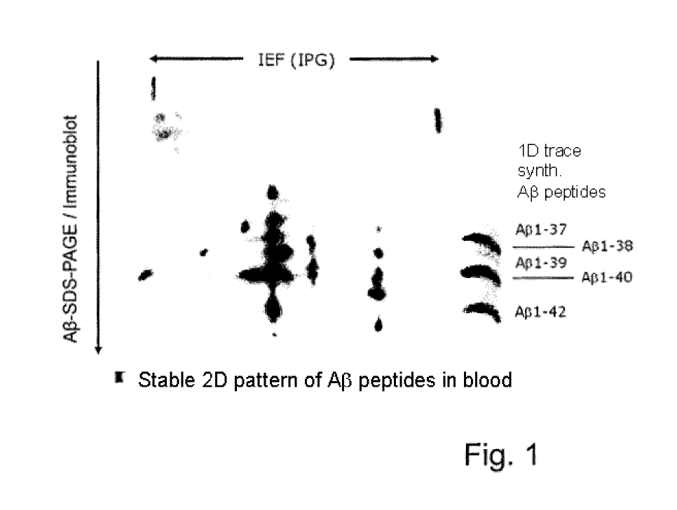

FIG. 1 shows an illustrative two-dimensional separation and labeling of A beta-peptides in blood based on a photograph of a 2D-Aβ-WIB. The figure shows a stable 2D pattern of the corresponding blood A beta-peptides.

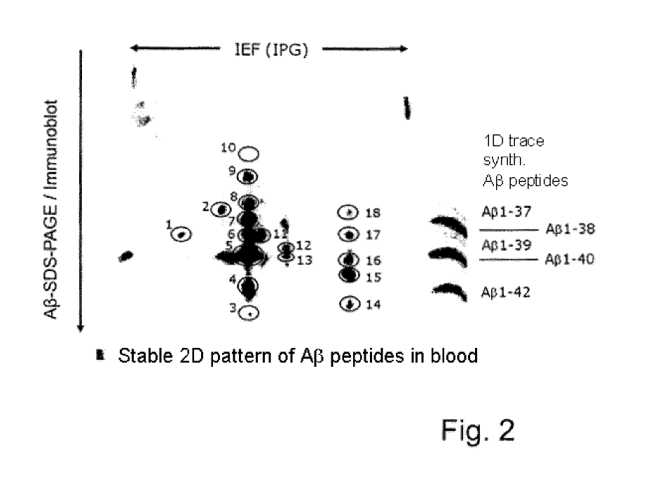

FIG. 2 shows a freely selected designation or numbering of a total of 18 amyloid beta-peptides separated by means of 2D-Aβ-WIB from blood plasma. More particularly, FIG. 2 shows an illustrative 2D-Aβ-WIB with the corresponding breakdown of the corresponding fields (spots) of the amyloid beta-peptides 1 to 18. The 18 fields are present in each of the membranes of the 2D blots studied or analyzed (n=42). Synthetic amyloid beta-peptides 1-37/38/39/40/42 are separated in the 1D line (STD), which shows that their migration position is not aligned precisely in the second dimension. In addition, FIG. 2 shows the alignment or positioning of points or fields of human amyloid beta-peptides: (4) Aβ(1-42); (5) Aβ(1-40); (6) Aβ(1-39); (7) Aβ(1-38); (8) Aβ(1-37); (14) Aβ(2-42); (15) Aβ(2-40); (16) Aβ(2-39); (17) Aβ(2-38); (18) Aβ(2-37). The identity of the Aβ-immunoreactive fields (1) and (2), and also (10) to (13), is not studied any further.

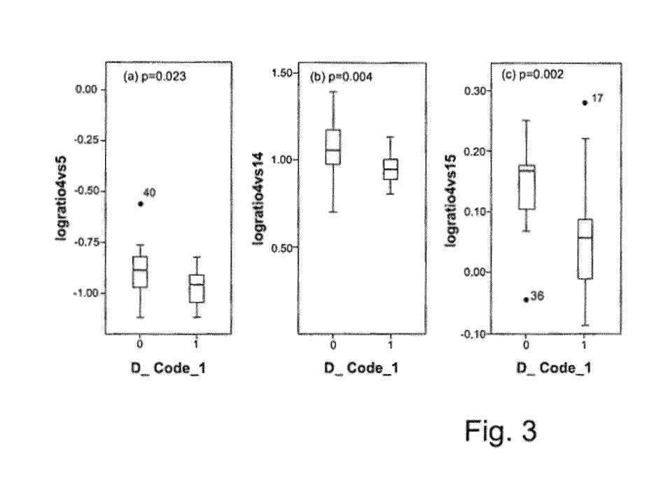

FIG. 3 shows a comparison of A beta-peptide ratios (a) 4/15 (Aβ(1-42)/Aβ(2-40)), (b) 4/14 (Aβ(1-42)/Aβ(2-42)) and (c) 4/5 (Aβ(1-42)/Aβ(1-40)) in the population of the overall study (n=42), with classification or grading into patients having early/incipient AD (eiAD; D_Code_{—}1=1) and a control (Con; D_Code_{—}1=0) according to the clinical gold standard (diagram in the form of fields (box plots); Mann-Whitney U test with statement of significance or meaningfulness on both sides).

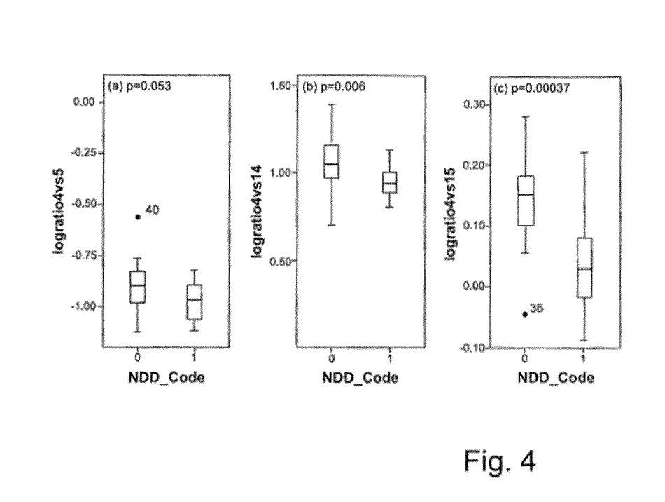

FIG. 4 shows a comparison of the A beta-peptide ratios (a) 4/5 (Aβ(1-42)/Aβ(1-40)), (b) 4/14 (Aβ(1-42)/Aβ(2-42)), and 4/15 (Aβ(1-42)/Aβ(2-40)) for a population examined, which is divided or graded into a group having eiAD (NDD_Code=1, n=18) and a group Con (NDD_Code=0, n=24) according to the neurochemical gold standard (diagram in the form of fields (box plots); Mann-Whitney U test with statement of significance or meaningfulness on both sides).

FIG. 5 shows a comparative ROC analysis for the A beta-peptide ratios 4/5 (Aβ(1-4)/Aβ(1-40)), 4/14 (Aβ(1-42)/Aβ(2-42)), and 4/15 (Aβ(1-42)/Aβ(2-40)) in patients having early/incipient AD (eiAD, n=21, D_Code_{—}1=1) versus control (Con, n=21, D_Code_{—}1=0), which are divided or graded according to the clinical gold standard. The ROC analysis is set to the identification of “true AD”, i.e., with D_Code_{—}1=1.

FIG. 5 is specified further by the following table:
Legend for FIG. 5: area under the graph
(ROC graph for D_Code_1 = 1, n = xx)

| Variables (n) | | Standard | Asymptotic |
|---|---|---|---|
| for test results | Area | deviation^{a} | significance^{b} |
| logratio4vs5 | 0.705 | 0.082 | 0.023 |
| logratio4vs14 | 0.760 | 0.076 | 0.004 |
| logratio4vs15 | 0.780 | 0.078 | 0.002 |

^{a}for non-parametric condition
^{b}zero hypothesis: “true area” = 0.5

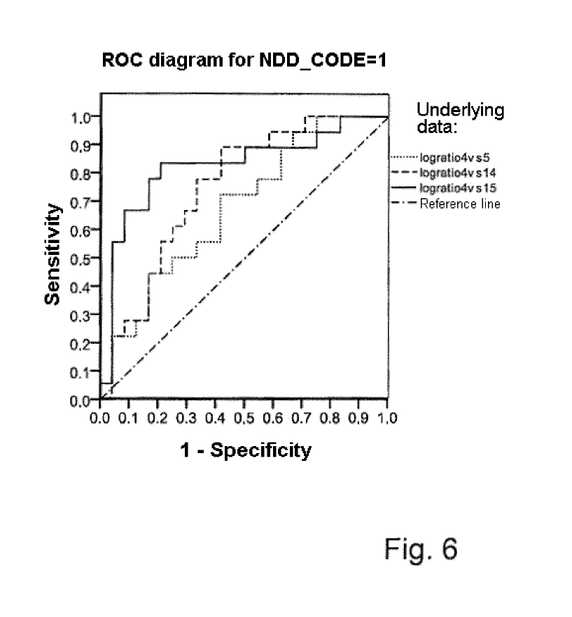

FIG. 6 shows a comparative ROC analysis for A beta-peptide ratios 4/5 (Aβ(1-42)/Aβ(1-40)), 4/14 (Aβ(1-42)/Aβ(2-42)), and 4/15 (Aβ(1-42)/Aβ(2-40)) in patients having early/incipient AD (eiAD, n=18, NDD_Code=1) versus control (Con, n=24, NDD_Code=0), divided or determined according to the neurochemical gold standard. The ROC analysis is set or adjusted to the identification of “true AD”, i.e., D_Code_{—}1=1.

FIG. 6 is specified further by the following table:
Legend for FIG. 6: area under the graph
(ROC graph for NDD_Code = 1, n = 18)

| Variables (n) | | Standard | Asymptotic |
|---|---|---|---|
| for test results | Area | deviation^{a} | significance^{b} |
| logratio4vs5 | 0.676 | 0.083 | 0.053 |
| logratio4vs14 | 0.750 | 0.075 | 0.006 |
| logratio4vs15 | 0.824 | 0.071 | 0.00037 |

^{a}for non-parametric condition
^{b}zero hypothesis: “true area” = 0.5

The ROC analysis (NDD_Code=1) results, for a given or fixed sensitivity of 83.3%, in specificities of 79.2% (cut-off value: 0.0938), 58.3% (cut-off value: 1.0296) and 37.5% (cut-off value: −0.8737 for logratio4vs15, logratio4vs14, and logratio4vs5).

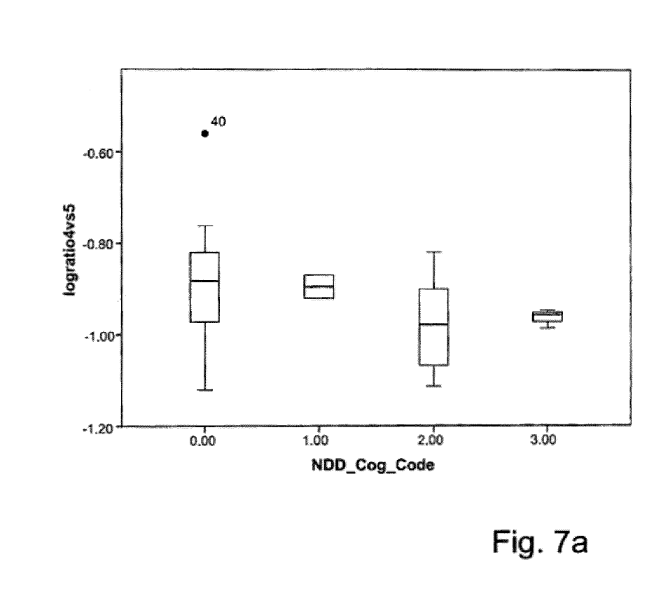

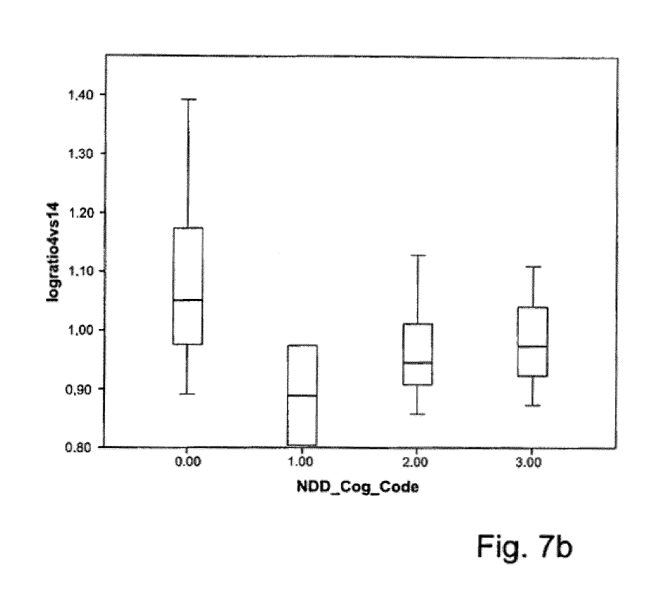

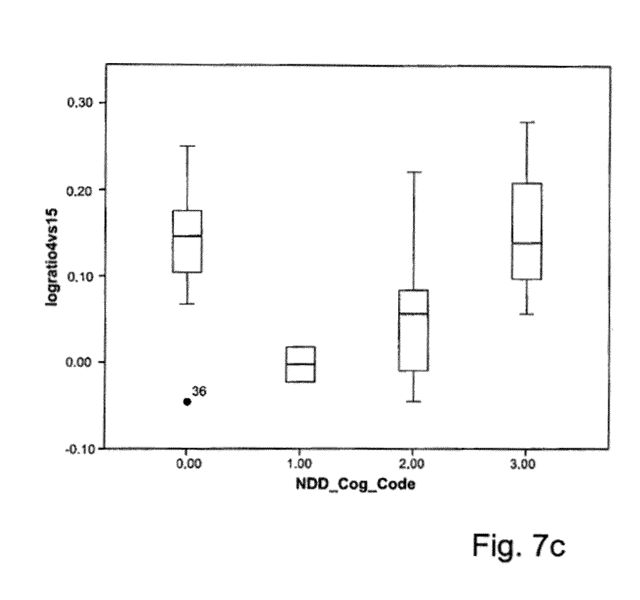

FIG. 7 shows a comparative analysis based on a diagram in the form of fields (box plot) with a non-parametric evaluation of significance or meaningfulness (Kruskal-Wallis-H) for (a) Aβ peptide ratios 4/5 (Aβ(1-42)/Aβ(1-40)), (b) 4/14 (Aβ(1-42)/Aβ(2-42)) and (c) 4/15 (Aβ(1-42)/Aβ(2-40)) for patients where there is a CSF dementia biomarker and a statement regarding performance in the context of a cognitive test based on an MMSE (n=38). The NDD_Cog_Code is used as a combined molecular and cognitive gold standard for patient classification:
NDD_Cog_Code=3:other dementias (oD); MMSE<25 and AvNDD_Score<1.0; (n=3);
NDD_Cog_Code=2: early Alzheimer's disease (eAD); MMSE<25 and AvNDD_Score≧1.0; (n=15);
NDD_Cog_Code=1:incipient Alzheimer's disease (iAD); MMSE 25-30 and AvNDD_Score≧1.0; (n=2); and
NDD_Cog_Code=0:no Alzheimer's disease (non-AD or noAD); MMSE 25-30 and AvNDD_Score<1.0; (n=18).

FIG. 7a shows the specific A beta-peptide ratio 4/5 (Aβ(1-42)/Aβ(1-40)); log values; significance value: p=0.132.

FIG. 7b shows the specific A beta-peptide ratio 4/14 (Aβ(1-42)/Aβ(2-42)); log values; significance value: p=0.027.

FIG. 7c shows the specific A beta-peptide ratio 4/15 (Aβ(1-42)/Aβ(2-40)); log values; significance value: p=0.013.

The figures which follow each show a scatterplot of the avNDD_Score versus (a) Aβ ratios 4/5 (Aβ(1-42)/Aβ(1-40)), (b) 4/14 (Aβ(1-42)/Aβ(2-42)) and (c) 4/15 (Aβ(1-42)/Aβ(2-40)). Specifically, the figures show:

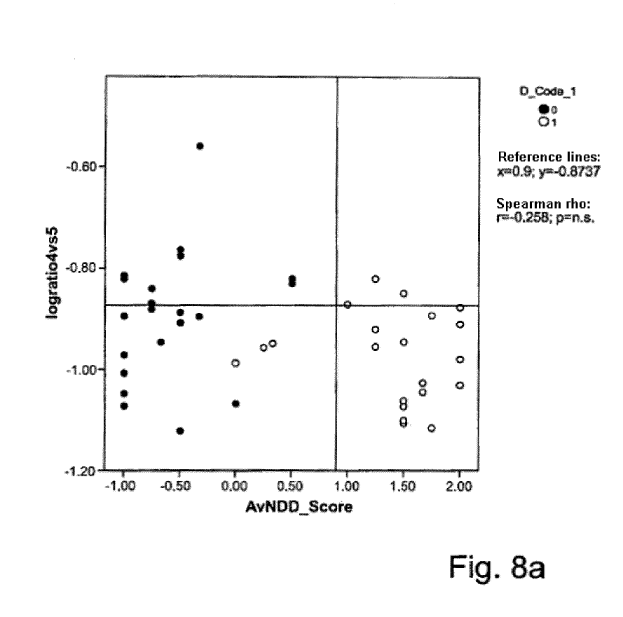

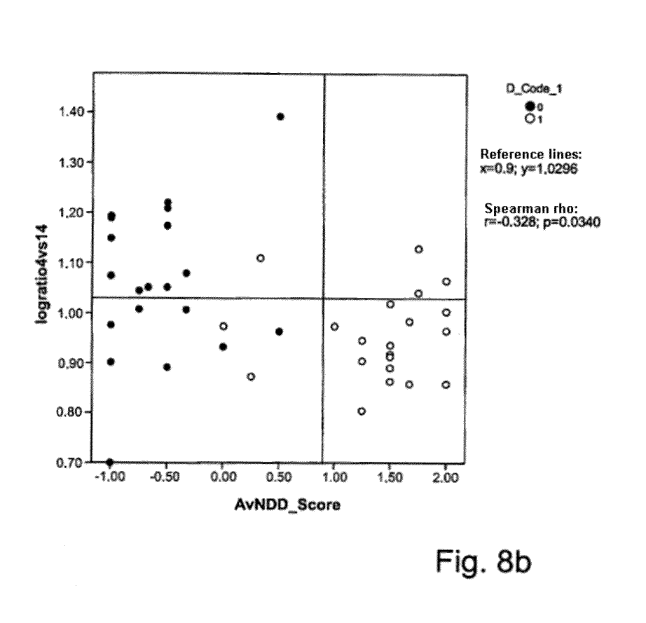

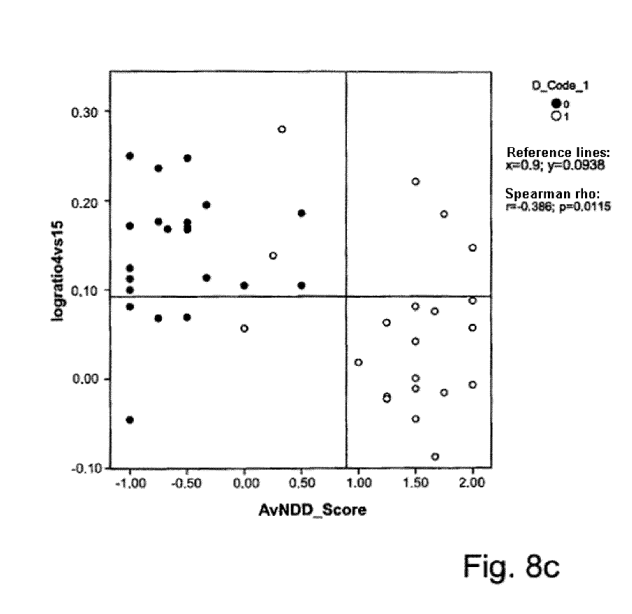

FIG. 8a shows a corresponding scatterplot where the cut-off line y=−0.8737 originates from the ROC analysis for log 4vs5, NDD_Code=1 (cf. FIG. 6); the AvNDD_Score cut-off line is freely set to x=0.9, since an AvNDD score≧1.0 assigns a risk of early or incipient AD (i.e., NDD_Code=1) to a subject or test person.

FIG. 8b shows a further scatterplot where the cut-off line y=1.0296 originates from the ROC analysis for log 4vs 14, NDD_Code=1 (cf. FIG. 6); the AvNDD_Score cut-off line is freely set to x=0.9, since an AvNDD score≧1.0 assigns a risk of early or incipient AD (i.e., NDD_Code=1) to a subject or test person.

FIG. 8c shows yet a further scatterplot where the cut-off line y=0.0938 originates from an ROC analysis for log 4vs15, NDD_Code=1 (cf. FIG. 6); the AvNDD_Score cut-off line is freely set to x=0.9, since an AvNDD score≧1.0 assigns a risk of early or incipient AD (i.e., NDD_Code=1) to a subject or test person.

The figures which follow are each scatterplots of the NDD_SPECT_Score versus A beta ratios (a) 4/5 (Aβ(1-42)/Aβ(1-40)), (b) 4/14 (Aβ(1-42)/Aβ(2-42)) and (c) 4/15 (Aβ(1-42)/Aβ(2-40)); for a group size of n=37. The individual figures show:

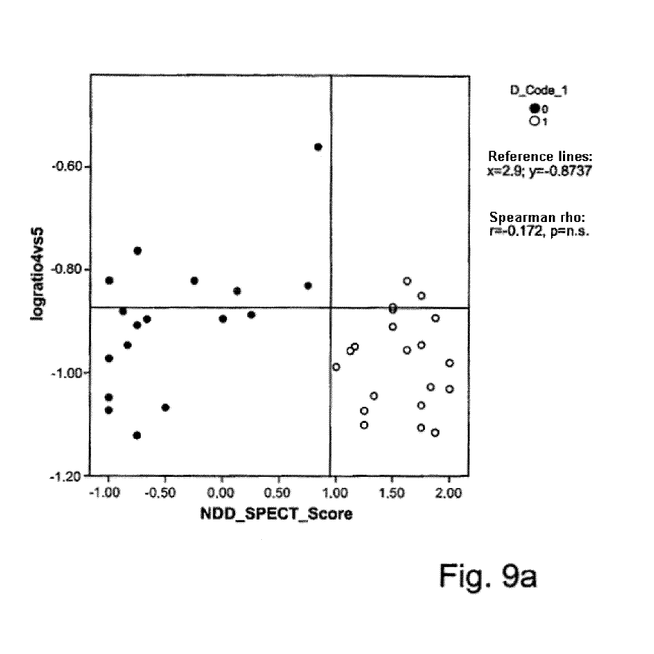

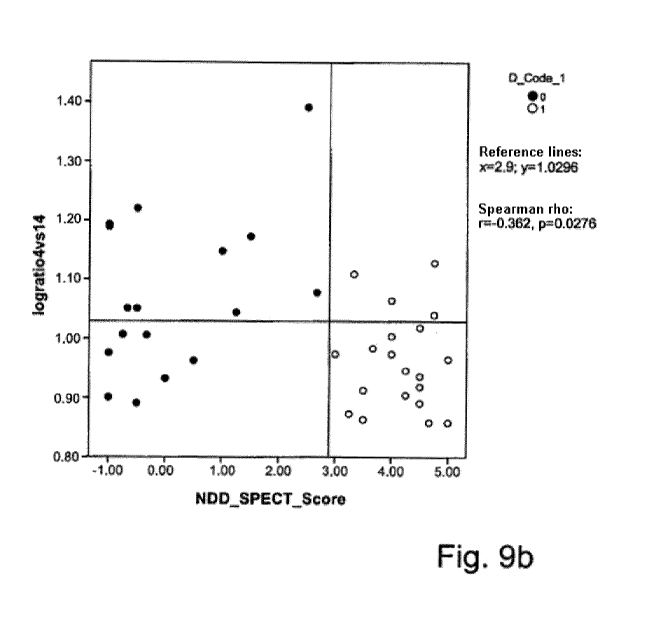

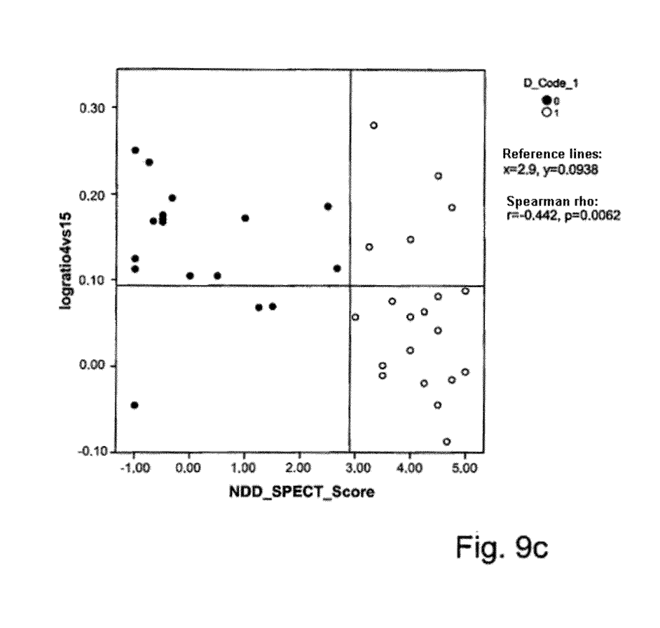

FIG. 9a shows a corresponding scatterplot where the cut-off line y=−0.8737 originates from the ROC analysis for log 4vs5, NDD_Code=1 (cf. FIG. 6); the NDD_SPECT_Score cut-off line is freely set to x=2.9, since, for an NDD_SPECT_Score≧3.0, patients assigned or classified according to the clinical gold standard can be separated or differentiated without overlap.

FIG. 9b shows a further scatterplot where the cut-off line y=1.0296 originates from the ROC analysis for log 4vs 14, NDD_Code=1 (cf. FIG. 6); the NDD_SPECT_Score cut-off line is freely set to x=2.9, since, for an NDD_SPECT_Score≧3.0, patients assigned or classified according to the clinical gold standard can be separated or differentiated without overlap.

FIG. 9c shows a corresponding scatterplot where the cut-off line y=0.0938 originates from the ROC analysis for log 4vs15, NDD_Code=1 (cf. FIG. 6); the NDD_SPECT_Score cut-off line is freely set to x=2.9, since, for an NDD_SPECT_Score≧3.0, patients assigned or classified according to the clinical gold standard can be separated or differentiated without overlap.

TABLE 3
Correlation matrix (Spearman rho; significance on both sides) of A beta-peptide
ratios in the blood, CSF dementia biomarkers, age, diagnostic gold standard,
cognitive parameters, neuroimaging data and ApoE-epsilon-4 genotype

| | | | logratio | logratio | logratio | | |
|---|---|---|---|---|---|---|---|
| | | | 4vs5 | 4vs14 | 4 vs15 | AvNDD_Score | NDD_SPECT_Score |
| logratio4vs5 | corr. | coeff. | 1.000 | .472** | .392* | −.258 | −.172 |
| | sig. | (2- | . | 0.00160 | 0.01022 | 0.09953 | 0.30811 |
| | n | | 42 | 42 | 42 | 42 | 37 |
| logratio4vs14 | corr. | coeff. | 0.472** | 1.000 | 0.593** | −0.328* | −0.362* |
| | sig. | (2- | 0.00160 | . | 0.00003 | 0.03399 | 0.02758 |
| | n | | 42 | 42 | 42 | 42 | 37 |
| logratio4vs15 | corr. | coeff. | 0.392* | 0.593** | 1.000 | −0.386* | −0.442** |
| | sig. | (2- | 0.01022 | 0.00003 | . | 0.01153 | 0.00619 |
| | n | | 42 | 42 | 42 | 42 | 37 |
| AvNDD_Score | corr. | coeff. | −0.258 | −0.328* | −0.386* | 1.000 | 0.904** |
| | sig. | (2- | 0.09953 | 0.03399 | 0.01153 | . | 0.00000 |
| | n | | 42 | 42 | 42 | 42 | 37 |
| NDD_SPECT_Score | corr. | coeff. | −0.172 | −0.362* | −0.442** | 0.904** | 1.000 |
| | sig. | (2- | 0.30811 | 0.02758 | 0.00619 | 0.00000 | . |
| | n | | 37 | 37 | 37 | 37 | 37 |
| logAGE | corr. | coeff. | 0.374* | −0.456** | 0.540** | 0.397** | 0.512** |
| | sig. | (2- | 0.01473 | 0.00241 | 0.00023 | 0.00922 | 0.00120 |
| | n | | 42 | 42 | 42 | 42 | 37 |
| Gender | corr. | coeff. | 0.212 | 0.071 | −0.216 | −0.101 | −0.107 |
| | sig. | (2- | 0.17690 | 0.65594 | 0.16883 | 0.52514 | 0.53020 |
| | n | | 42 | 42 | 42 | 42 | 37 |
| ApoE_Code_1 | corr. | coeff. | −0.251 | −0.282 | −0.430** | 0.391* | 0.310 |
| | sig. | (2- | 0.10857 | 0.07001 | 0.00452 | 0.01045 | 0.06185 |
| | n | | 42 | 42 | 42 | 42 | 37 |
| logAβx40TGC | corr. | coeff. | 0.146 | −0.149 | −0.289 | 0.223 | 0.312 |
| | sig. | (2- | 0.46772 | 0.45836 | 0.14407 | 0.26322 | 0.14751 |
| | n | | 27 | 27 | 27 | 27 | 23 |
| logAβx42TGC | corr. | coeff. | 0.254 | −0.075 | −0.203 | −0.585** | −0.628** |
| | sig. | (2- | 0.27960 | 0.75273 | 0.39067 | 0.00674 | 0.00916 |
| | n | | 20 | 20 | 20 | 20 | 16 |
| logAβ142Innx | corr. | coeff. | 0.215 | 0.289 | 0.226 | −0.844** | −0.736** |
| | sig. | (2- | 0.17184 | 0.06312 | 0.14962 | 0.00000 | 0.00000 |
| | n | | 42 | 42 | 42 | 42 | 37 |
| logAβratioTGC | corr. | coeff. | 0.286 | 0.122 | 0.075 | −0.878** | −0.875** |
| | sig. | (2- | 0.22203 | 0.60896 | 0.75273 | 0.00000 | 0.00001 |
| | n | | 20 | 20 | 20 | 20 | 16 |
| logCSFratio | corr. | coeff. | 0.131 | 0.404* | 0.366 | −0.786** | −0.788** |
| Aβ142vsAβ40 | sig. | (2- | 0.51402 | 0.03686 | 0.06068 | 0.00000 | 0.00001 |
| | n | | 27 | 27 | 27 | 27 | 23 |
| logTotalTau | corr. | coeff. | −0.247 | −0.300 | −0.438** | 0.809** | 0.787** |
| | sig. | (2- | 0.11425 | 0.05367 | 0.00376 | 0.00000 | 0.00000 |
| | n | | 42 | 42 | 42 | 42 | 37 |
| logPhospho Tau181 | corr. | coeff. | −0.375* | −0.471** | −0.343 | 0.866** | 0.875** |
| | sig. | (2- | 0.04480 | 0.00984 | 0.06864 | 0.00000 | 0.00000 |
| | n | | 29 | 29 | 29 | 29 | 25 |
| logMMSE | corr. | coeff. | 0.464** | 0.270 | 0.254 | −0.728** | −0.711** |
| | sig. | (2- | 0.00332 | 0.10133 | 0.12369 | 0.00000 | 0.00000 |
| | n | | 38 | 38 | 38 | 38 | 35 |
| NDD_Cog_Code | corr. | coeff. | −0.369* | −0.417** | −0.315 | 0.749** | 0.760** |
| | sig. | (2- | 0.02254 | 0.00917 | 0.05379 | 0.00000 | 0.00000 |
| | n | | 38 | 38 | 38 | 38 | 35 |
| MRI_Score | corr. | coeff. | −0.238 | −0.369* | −0.308 | 0.540** | 0.590** |
| | sig. | (2- | 0.14413 | 0.02072 | 0.05683 | 0.00039 | 0.00015 |
| | n | | 39 | 39 | 39 | 39 | 36 |
| SPECT_Score | corr. | coeff. | −0.067 | −0.281 | −0.376* | 0.640** | 0.868** |
| | sig. | (2- | 0.69445 | 0.09261 | 0.02176 | 0.00002 | 0.00000 |
| | n | | 37 | 37 | 37 | 37 | 37 |

The present invention is not limited to embodiments described herein; reference should be had to the appended claims.

What is claimed is:

1-36. (canceled)

37. A method of using at least one quantitative ratio of two different amyloid beta-peptides in a sample of a body fluid from a patient for determining the patient's probability of contracting Alzheimer's disease or for determining the patient's suffering from a precursor of Alzheimer's disease, the method comprising:
obtaining the sample of the body fluid from the patient;
calculating the at least one quantitative ratio from the two different amyloid beta-peptides from the sample; and
determining the patient's probability of contracting Alzheimer's disease (AD) or the patient's suffering from a precursor of Alzheimer's disease using the at least one quantitative ratio,
wherein, the two different amyloid beta-peptides are selected from (a) Aβ(1-42), (b) Aβ(2-40) and (c) Aβ(2-42), and
the at least one quantitative ratio is selected from (a) Aβ(1-42)/(b) Aβ(2-40), (a) AB (1-42)/(c) Aβ(2-42), (b) Aβ(2-40)/(a) Aβ(1-42) and (c) Aβ(2-42)/(a) Aβ(1-42).

38. The method as recited in claim 37, wherein the at least one quantitative ratio is calculated from (a) Aβ(1-42)/(b) Aβ(2-40) or from (a) Aβ(1-42)/(c) Aβ(2-42).

39. The method as recited in claim 37, wherein the sample is blood.

40. The method as recited in claim 37, further comprising:
removing and isolating the amyloid beta-peptides (a) Aβ(1-42), on the one hand, and at least one of (b) Aβ(2-40) or (c) Aβ(2-42), on the other hand, from the sample via an immunoprecipitation.

41. The method as recited in claim 37, further comprising:
comparing the at least one quantitative ratio of the two different amyloid beta-peptides with corresponding reference ratios or assigning the at least one quantitative ratio of the two different amyloid beta-peptides to the corresponding reference ratios, wherein the comparing of the at least one quantitative ratio with the corresponding reference ratios is used to determine the probability of contracting Alzheimer's disease or to determine the patient's suffering from the precursor of Alzheimer's disease, or
determining the at least one quantitative reference ratio based on a reference group of patients.

42. The method as recited in claim 37, further comprising at least one of:
assigning the patient an elevated probability of contracting Alzheimer's disease;
determining that the patient is suffering from a preclinical stage of Alzheimer's disease; and
determining that the patent is suffering from a clinical precursor of Alzheimer's disease, when an amount of at least one of (b) Aβ(2-40) and (c) Aβ(2-42) is elevated compared to a corresponding amount from a reference group not having diagnosed Alzheimer's disease or not having a diagnosed precursor of Alzheimer's disease, and at least one of the ratios of (a) AB (1-42)/(b) Aβ(2-40) and of (a) Aβ(1-42)/(c) Aβ(2-42) is reduced.

43. The method as recited in claim 37, further comprising
assigning the patient an elevated probability of contracting Alzheimer's disease, or
determining that the patient is suffering from a preclinical stage of Alzheimer's disease, or
determining that the patient is suffering from a clinical precursor of Alzheimer's disease, when a relative ratio of Aβ(1-42)/Aβ(2 y) is reduced compared to corresponding reference ratios for a reference group not having diagnosed Alzheimer's disease or not having a diagnosed precursor of Alzheimer's disease, where y is an integer from 37 to 43, or
when at least one of relative ratios of (a) Aβ(1-42)/(b) Aβ(2-40) and of (a) Aβ(1-42)/(c) Aβ(2-42) is reduced compared to the corresponding reference ratios for the reference group not having diagnosed Alzheimer's disease or not having the diagnosed precursor of Alzheimer's disease.

44. A method for determining a probability of a patient contracting Alzheimer's disease, the method comprising:
determining at least one quantitative ratio of two different amyloid beta-peptides in a sample of a body fluid from the patient,
wherein, the two different amyloid beta-peptides are selected from (a) Aβ(1-42), (b) Aβ(2-40) and (c) Aβ(2-42), and
the at least one quantitative ratio is formed from (a) Aβ(1-42)/(b) Aβ(2-40), (a) Aβ(1-42)/(c) Aβ(2-42), (b) Aβ(2-40)/(a) Aβ(1-42) and (c) Aβ(2-42)/(a) Aβ(1-42).

45. A method for determining a probability of a patient contracting Alzheimer's disease, the method comprising:
determining at least one quantitative ratio of two different amyloid beta-peptides in a sample of a body fluid from the patient,
comparing the at least one quantitative ratio with corresponding reference ratios or assigning the at least one quantitative ratio to the corresponding reference ratios; and
determining a probability of the patient contracting Alzheimer's disease based on the comparison
wherein, the two different amyloid beta-peptides are selected from (a) Aβ(1-42), (b) Aβ(2-40) and (c) Aβ(2-42), and
the at least one quantitative ratio is selected from (a) Aβ(1-42)/(b) Aβ(2-40), (a) Aβ(1-42)/(c) Aβ(2-42), (b) Aβ(2-40/(a) Aβ(1-42) and (c) Aβ(2-42)/(b) Aβ(2-40).

46. A kit for determining a probability of a patient contracting Alzheimer's disease or for determining the patent's suffering from a precursor of Alzheimer's disease, the kit comprising:
components for determining a quantitative ratio of two different amyloid beta-peptides in a sample of a body fluid from the patient,
wherein, the components are selected so as to provide a quantitative determination of two different amyloid beta-peptides selected from (a) Aβ(1-42), (b) Aβ(2-40) and (c) Aβ(2-42) so as to determine at least one quantitative ratio selected from (a) Aβ(1-42)/(b) Aβ(2-40), (a) Aβ(1-42)/(c) Aβ(2-42), (b) Aβ(2-40)/(a) Aβ(1-42) and (c) Aβ(2-42)/(a) Aβ(1-42).
